(19) United States
(12) Patent Application Publication
(10) Pub. No.: US 2003/0190381 A1
(43) Pub. Date: Oct. 9, 2003

(54) Medical composition for balancing bodily processes

(76) Inventors: Jeffrey S. Bland, Fox Island, WA (US); DeAnn J. Liska, Tacoma, WA (US); Kim C. Krumhar, Carlsbad, CA (US); Matthew Tripp, Gig Harbor, WA (US); Gary K. Darland, Gig Harbor, WA (US); Robert Lerman, Gig Harbor, WA (US); Daniel O. Lukaczer, Gig Harbor, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/352,388

(22) Filed: Jan. 27, 2003

Related U.S. Application Data
(63) Continuation-in-part of application No. 10/056,858, filed on Jan. 23, 2002.
(60) Provisional application No. 60/265,908, filed on Feb. 2, 2001.
(60) Provisional application No. 60/352,016, filed on Jan. 25, 2002.
(60) Provisional application No. 60/432,689, filed on Dec. 11, 2002.

(51) Int. Cl. A61K 35/78

(52) U.S. Cl. 424/757; 424/725

(57) ABSTRACT

Medical compositions and methods using same to nutritionally support balance of bodily processes are disclosed. A medical composition to nutritionally support balance of bodily processes involving S-adenosylmethionine is disclosed.

### CROSS-REFERENCE TO RELATED APPLICATIONS

This application is a continuation-in-part of U.S. application Ser. No. 10/056,858, filed Jan. 23, 2002, which claims the benefit under 35 U.S.C. § 119(e) of U.S. Provisional Application No. 60/265,908, filed Feb. 2, 2001. This application claims the benefit under 35 U.S.C. § 119(e) of U.S. Provisional Application No. 60/352,016, filed Jan. 25, 2002; and U.S. Provisional Application No. 60/432,689, filed Dec. 11, 2002.

### BACKGROUND OF THE INVENTION

1. Field of the Invention

This invention relates to a medical composition. More particularly, this invention relates to a medical composition for providing a natural approach to managing bodily processes involving S-adenosylmethionine.

2. Description of the Related Art

A variety of nutritional approaches have been tried to manage premenstrual syndrome (PMS), a condition generally defined as symptoms occurring in the second half or luteal phase of the menstrual cycle. Research in this area has met with uneven success, and to date the underlying mechanism of these nutritional interventions has been poorly understood.

PMS is a condition whose cause is not completely clear. Symptoms generally involve, but not limited to, mood swings, headaches, bloating, water retention, and/or breast tenderness that occur in the second half of the monthly menstrual cycle. It is estimated that PMS afflict up to 40 percent of women of reproductive age, with severe effects that can compromise ability to perform daily tasks in five to ten percent of women.

Hormone Balance

It is well known that one of the causes of breast cancer, as well as many other hormone related health problems in both men and women, is excessive estrogen exposure from both endogenous and exogenous sources. Improving estrogen metabolism can be of benefit to women with various conditions and family histories, including, but not limited to, a family history of breast, uterine, or ovarian cancer; and conditions such as, but not limited to, endometriosis, premenstrual syndrome, uterine fibroid tumors, fibrocystic or painful breasts, cervical dysplasia, and systemic lupus erythematosis. Other conditions associated with hormone imbalance can include, but are not limited to, vaginitis, fatigue, cognitive dysfunction, depression, and irritability. Beneficial modulation of estrogen metabolism can be accomplished through dietary and lifestyle modifications, such as increasing fiber and reducing fat, increasing phytoestrogen intake, losing weight, and increasing exercise. In addition, many nutrients can effectively reduce estrogen load by supporting preferred pathway of estrogen metabolism and detoxification, including, but not limited to, indole-3-carbinol, B vitamins, magnesium, limonene, calcium D-glucarate, and antioxidants. The influences of these nutrients on estrogen metabolism can have profound significance for diseases in which these hormones can play a role in clinical expression.

The term “estrogen” is used to collectively describe the female hormones, the most potent of which is estradiol. The other estrogens are estrone and estriol. Estrogens affect the growth, differentiation, and function of diverse target tissues—not only those involved in the reproductive process, but tissues throughout the body. Estrogens can play an important role in bone formation and maintenance, exert cardioprotective effects, and influence behavior and mood. Although estrogen is best known for its critical role in female reproduction, less well-known roles are the important actions of estrogen in male tissues, such as the prostate and testes.

In women, estrogens can be synthesized from cholesterol in the ovaries in response to pituitary hormones. In an adult woman with normal cycles, the ovarian follicle secretes about 70 to 500 μg of estradiol per day, depending on the phase of the menstrual cycle. Estradiol can be converted to estrone and vice versa, and both can be converted to the major urinary metabolite, estriol. Estrogens can also be produced by the aromatization of androgens in fat cells, skin, bone, and other tissues. After menopause, most endogenous estrogen is produced in the peripheral tissues by the conversion of androstenedione, which is secreted by the adrenal cortex, to estrone. In addition, some estrogen continues to be manufactured by aromatase in body fat, and the ovaries continue to produce small amounts of the male hormone testosterone, which is converted to estradiol. The total estrogen produced after menopause, however, is far less than that produced during a woman's reproductive years.

Estradiol and other naturally occurring estrogens circulate in the body bound mainly to the sex hormone binding globulin (SHBG); however, unbound estrogens can enter target-tissue cells and induce biological activity. Accordingly, any change in the concentration of can alter estrogen metabolism by inducing changes in the availability of estrogen to the target cell.

Estrogen Metabolism and Detoxification

Metabolism of estrogen within the body is a complex subject. Estrone and estradiol are biochemically interconvertible and yield substantially the same family of estrogen metabolites. Because these metabolites vary greatly in biological activity, the ultimate biologic effect of estrogen depends on how it is metabolized. The metabolism of estrogen takes place primarily in the liver through Phase I (hydroxylation) and Phase II (methylation, glucuronidation, and sulfation) pathways with ultimate excretion in urine and feces.

Hydroxylation

Cytochrome P-450 enzymes mediate the hydroxylation of estradiol and estrone, which is the major Phase I metabolic pathway for endogenous estrogens. This reaction takes place at two primary sites on the estrogen molecule, either at the 2 carbon (C-2) position yielding 2-hydroxyestrone (2-OH) or at the 16α carbon (C-16α) position yielding 16α-hydroxyestrone (16α-OH). Another contribution is made from hydroxylation at the 4 carbon (C-4) position yielding 4-hydroxyestrone (4-OH). The 2-OH metabolite confers weak estrogenic activity, and is generally termed the “good” estrogen. In contrast, the 16α-OH and 4-OH metabolites show persistent estrogenic activity and promote tissue proliferation. It is suggested that women who metabolize a larger proportion of their endogenous estrogen via the C-16α hydroxylation pathway can be at significantly elevated risk of breast cancer compared with women who metabolize proportionally more estrogen via the C-2 pathway.

Methylation

The 2-OH and 4-OH metabolites (catechol estrogens) can be readily oxidized to quinones, which are reactive and can damage DNA and promote carcinogenesis directly or indirectly through the generation of reactive oxygen species. This harmful pathway can be minimized through preferential detoxification and excretion of the catechol estrogens via Phase II methylation by the catechol-O-methyltransferase (COMT) enzyme. This methylation requires S-adenosylmethionine (SAM) and magnesium as cofactors. COMT is present in most tissues and converts catechols into their corresponding methyl ester metabolites, which are more water-soluble. Recent data suggest that the methylation of 4-OH renders this harmful metabolite significantly less active, while 2-methoxyestrone can manifest beneficial properties by inhibiting breast cancer.

Methylenetetrahydrofolate reductase (MTHFR) is an enzyme in the control of the folate cycle and methylation. A polymorphism in the MTHFR gene can be found in a certain percentage of the population. One effect of the polymorphism in the MTHFR gene can be expressed as a protein that can affect the levels of S-adenosylmethionine (SAM), which is a cofactor used for methylation of compounds. With lowered levels of SAM, methylation of estrogen can also be lowered in women with the certain polymorphism. Accordingly, women with the certain polymorphism have a higher risk of conditions associated with high levels of estrogen.

Glucuronidation

Glucuronidation is one of the Phase II liver detoxification pathways for estrogens and other toxins. Glucuronic acid is conjugated with the estrogen to facilitate its elimination from the body. Unfortunately, some intestinal bacteria (mostly pathogenic) possess an enzyme, β-glucuronidase, that can uncouple the bond between excreted estrogen and glucuronic acid in the large intestine, allowing the estrogen to reenter circulation (enterohepatic recirculation). Accordingly, excess β-glucuronidase activity is associated with an increased cancer risk, including breast cancer among others. The activity of β-glucuronidase can be increased when the diet is high in fat and low in fiber and can be reduced by establishing a proper bacterial flora by eating a diet high in plant foods and supplementing the diet with the “friendly bacteria”, such as, but not limited to, Lactobacillus acidophiius and Bifidobacterium infantis.

Sulfation

Another Phase II liver detoxification pathway for estrogens and other toxins is sulfation. Sulfation of estrogen and estrogen metabolites can occur with the aid of N-acetylcysteine. Sulfation can be a route of elimination of estrogenic compounds. However, the 2-OH form metabolite is preferentially sulfated and sulfation has been shown to increase storage of catechol estrogens.

Estrogen Receptors

Estrogens, like all steroid hormones, can have a wide range of actions and affect almost all systems in the body, yet act in a tissue-specific manner. Estrogens can act by binding with high affinity to the estrogen receptor (ER) in target cells. Once bound by estrogens, the receptor undergoes a conformational change and binds to specific DNA sequences. This transcription complex can regulate the expression of target genes within a cell. Because the ER has a unique ability to bind with a wide variety of compounds with diverse structural features, many environmental toxins and plant compounds can bind to the ER with varying affinities and modulate estrogen activity.

Two forms of the estrogen receptor, α and β, have been identified that differ in tissue distribution, binding affinity, and biological function. Therefore, different target cells can respond differently to the same estrogenic stimulus depending on the ratio of expression of the two receptor subtypes in the cell. Therefore, phytoestrogens and new designer estrogen drugs, such as tamoxifen and taloxifene, called selective estrogen receptor modulators (SERMs) can behave like estrogens in some tissues, but block its action in others.

Estrogen and Cancer

Epidemiological and animal studies have identified estrogen exposure as a risk factor for several cancers, namely breast, endometrium, ovary, prostate, testis, and thyroid among others. Much of the evidence comes from the observation that cancer risk increases with increased exposure to endogenous or exogenous estrogens and the positive relationship observed between blood levels of estrogens and cancer risk. Prolonged estrogen exposure can cause direct genotoxic effects by inducing cell proliferation in estrogen-dependent target cells (increasing the opportunity for the accumulation of random genetic errors), affecting cellular differentiation, and altering gene expression. Additionally, there is increasing evidence for indirect genotoxic effects of estrogens, as well. The relative importance of each mechanism is likely a function of the specific estrogen, as well as the exposed tissue or cell type and its metabolic state.

Direct Genotoxic Effects

Evidence is accumulating that certain estrogen metabolites can be directly responsible for the initial genetic damage leading to tumors. 16α-OH and 4-OH are estrogen metabolites that have been associated with direct genotoxic effects and carcinogenicity. Some researchers believe increased levels of 16α-OH can increase the risk of breast cancer by increasing both cell proliferation and direct DNA damage; however, scientific consensus has not yet been reached. Conversely, 2-OH can induce apoptosis and thereby inhibit cell proliferation, a mechanism in the prevention of cancer.

A recent 5-year prospective study of 10,786 women was conducted to investigate the role of estrogen metabolism as a predictor of breast cancer, specifically the ratio of 2-OH to 16α-OH. The researchers found that premenopausal women who developed breast cancer had a decreased 2-OH: 16α-OH ratio and a higher percentage of 16α-OH than 2-OH. Women with predominately 2-OH were 40% less likely to have developed breast cancer during the 5 years. Another recent case-control study that began in 1977 found that postmenopausal women who developed breast cancer had a 15% lower 2-OH: 16α-OH ratio than control subjects. Furthermore, those with the highest 2-OH: 16α-OH ratios had about a 30% lower risk to breast cancer than women with lower ratios.

Diverse factors can add to the hormonal risk by decreasing the 2-OH: 16α-OH ratio, including, but not limited to, numerous pesticides and carcinogens, certain drugs, such as cyclosporin and cimetidine (Tagamet), obesity, and genetic predisposition. Dietary interventions, such as increased consumption of cruciferous vegetables (e.g., broccoli and cabbage) and phytoestrogen-rich foods, such as, but not limited to, soy and flaxseeds can significantly promote C-2 hydroxylation and increase the 2-OH: 16α-OH ratio.

Indirect Genotoxic Effects

Excessive production of reactive oxygen species has been reported in breast cancer tissue, and free-radical toxicity, which manifests as DNA single-strand breaks, lipid peroxidation, and chromosomal abnormalities, has been reported in hamsters treated with estradiol. The oxidation of catechol estrogens (2-OH and 4-OH) can yield reactive molecules called quinones. Quinones are thought to play a role in carcinogenesis by inducing DNA damage directly or as a result of redox cycling between the quinones and their semiquinone radicals, which generates reactive oxygen species, including superoxide, hydrogen peroxide, hydroxyl radicals, and the like. Supplementation with antioxidant nutrients can reduce the oxidation of the catechols and promote greater excretion of these metabolites through the methylation pathway.

Risk Factors for Increased Estrogen Exposure

There are many lifestyle factors that can influence the body's production of estrogen. Obesity can increase endogenous estrogen production by fat tissue, where the enzyme aromatase converts adrenal hormones into estrogen. Excess insulin in the bloodstream can prompt the ovaries to secrete excess testosterone and reduce SHBG levels, thus increasing levels of free estrogen. Alcohol consumption can increase estrogen levels, and epidemiological studies suggest that moderate alcohol consumption can increase the risk of breast cancer, an effect that may be synergistically enhanced when combined with estrogen replacement therapy.

Two sources of exogenous estrogens are oral contraceptives and hormone replacement therapy. Another source is environmental toxins that are structurally similar to estrogen and have the ability to mimic harmful estrogens in the body. These include aromatic hydrocarbons and organochlorines found in pesticides, herbicides, plastics, refrigerants, industrial solvents, and the like. Furthermore, the hormones used to fatten livestock and promote milk production can be unknowingly ingested when consuming meat and milk products, thereby increasing exposure to environmental estrogens.

While these lifestyle and environmental factors can influence the hormone burden of an individual, endogenous hormone levels can also have a genetic basis that can be a risk factor for hormone-dependent cancers and other conditions. Family history can be an indicator of potential problems in this area.

As shown in Table 1, sources of estrogens—whether environmental, dietary, or endogenously produced—can affect ER function. These substances can bind to estrogen α or β receptors with varying affinities and for varying lengths of time, producing a wide range of estrogen-related effects.

TABLE 1
Sources of Estrogens

| | Dietary Estrogens | |
|---|---|---|
| Environmental Estrogens | (“Phytoestrogens”) | Endogenous Estrogens |
| Organochlorine chemicals, | Isoflavones (e.g., genistein, | Estradiol |
| such as vinyl chlorides, | daidzein, equol, puerarin, | |
| dioxins, PCBs, and | coumestrol, glycitein, | |
| perchloroethylent (˜half of | biochanins) (from soy, beans, | |
| “endocrine disrupters” are in | peas, clover, alfalfa, and | |
| this class.) | kudzu) | |
| Non-organochlorine | Lignans (e.g., matairesinol, | Estrone |
| chemicals, such as phthalates | pinoresinol, | |
| and phenols (plasticizers), | secoisolariciresinol) | |
| aromatic hydrocarbons, and | (especially from flaxseed, | |
| some surfactants | rye, wheat, and sea | |
| | vegetables) | |
| Medications, such as | Certain flavenoids (e.g., | Estriol |
| hormone replacement, oral | rutin, naringenin, luteolin, | |
| contraceptives, tamoxifen, | resveratrol, quercetin) | |
| and cimetidine | (especially from citrus fruits | |
| | and grapes) | |
| Agricultural hormones in | | Hydroxylated estrogen |
| animal products consumed | | metabolites |
| by humans | | |
| | | Methoxylated estrogen |
| | | metabolites |
| | | Other estrogen metabolites |

Manifestations of Excessive Estrogen Exposure and Estrogen Dominance

An abundance of evidence indicates that excessive estrogen exposure from both endogenous and exogenous sources can be a causal factor in the development of cancer in hormone-dependent tissues, such as, but not limited to, breast, endometrium, ovary, uterus, and prostate. Furthermore, hormonal imbalances between progesterone, testosterone, and estrogen can lead to symptoms and conditions of estrogen dominance. These include premenstrual syndrome (PMS), endometriosis, uterine fibroid tumors, fibrocystic or painful breasts, cervical dysplasia, and systemic lupus erythematosis.

### SUMMARY OF THE INVENTION

The preferred embodiments provide a medical composition and a method of use thereof for promoting a healthy management of compounds in a body that involve methylation.

A certain embodiment provides method of managing a bodily process that utilizes S-adenosylmethionine (SAM) in a pathway of the bodily process comprising administering a composition comprising a mixture of an isoflavone, an isoflavone synergist, and a methylation support compound.

Another embodiment provides a method of treating or preventing a condition or disease involving a bodily process that utilizes S-adenosylmethionine (SAM) in a pathway of the bodily process comprising administering a composition comprising a mixture of an isoflavone, an isoflavone synergist, and a methylation support compound.

Another embodiment provides a method of treating hot flushes comprising administering a composition comprising a mixture of an isoflavone, an isoflavone synergist, and a methylation support compound.

It is preferable to use the medical composition to manage bodily processes that utilize SAM in the pathway. Hence, the medical composition can be used to affect a wide variety of bodily processes. The components of the medical composition can be varied accordingly to achieve a specific effect on a certain bodily process.

Other embodiments provide a method of use thereof for balancing estrogens in relation to other hormones that are involved in a woman's monthly cycle.

It is preferable to balance hormones by affecting the pathways of detoxification of estrogen and estrogenic metabolites. Mechanisms of action of detoxification of estrogen and estrogenic metabolites include promoting C-2 hydroxylation over C-4 and/or C-16α hydroxylation of estrogens, reducing oxidation of catechol estrogens (2-OH and 4-OH), promoting methylation of catechol estrogens (2-OH and 4-OH), increasing circulating concentrations of sex hormone binding globulin (SHBG), thus reducing levels of unbound, active estrogens, inhibiting activity of aromatase, which converts testosterone and androstenedione into estradiol and estrone, respectively, and promoting the detoxification of estrogens by upregulating Phase I and Phase II enzymes. It is more preferable that the mechanism of action to be affected is promoting methylation of catechol estrogens (2-OH and 4-OH).

### BRIEF DESCRIPTION OF THE DRAWINGS

FIG. 1 is a graph showing total scores for Shortened Premenstrual Assessment Form (SPAF) for subjects who completed the clinical study of Example 3.

FIG. 2 is a graph showing scores from representative categories of MDQ for all subjects who completed the clinical study of Example 3.

FIG. 3 is a graph showing quality-of-life assessment using an SF-36 questionnaire for subjects who completed the clinical study of Example 3.

FIG. 4 is a graph showing means for initial and final serum progesterone for twenty-six subjects who showed initial serum progesterone values of below 10 ng/mL in the clinical study of Example 3.

FIG. 5 is a graph showing means for initial and final sex hormone-binding globulin (SHBG) for twenty subjects who showed initial SHBG values of below 55 nmol/L in the clinical study of Example 3.

FIG. 6 is a graph showing an average number of hot flushes at the start (shaded bar) as compared to the average at the end (clear bar) for all participants completing the trial of Example 4.

FIG. 7 is a graph showing the results of the Greene Questionnaire with initial (shaded bars) and final (clear bars) scores for the subjects who completed the trial in the clinical study of Example 4.

FIG. 8 is a graph showing change in total cholesterol/HDL-cholesterol for all subjects who completed the trial in the clinical study of Example 4.

FIG. 9 is a graph showing change in total cholesterol/HDL-cholesterol over the 12 week intervention stratified between subjects who started with cholesterol/HDL-cholesterol <4 (clear bar) and >4 (shaded bar) in the clinical study of Example 4.

FIG. 10 is a graph showing change in blood homocysteine stratified by subjects who initially presented with homocysteine levels <8 pg/ml (clear bars)and >8 pg/ml (shaded bars) in the clinical study of Example 4.

FIG. 11 is a graph showing change in 16α-OH estrone for all subjects who finished the trial of the clinical study of Example 4.

FIG. 12 is a graph showing change in 2-OH estrone for all subjects who finished the trial of the clinical study of Example 4.

FIG. 13 is a graph showing change in 2-OH estrone/16α-OH estrone for all subjects who finished the trial of the clinical study of Example 4.

### DETAILED DESCRIPTION OF THE EMBODIMENTS

Before the present medical composition and method of use thereof are disclosed and described, it is to be understood that this invention is not limited to the particular configurations, process steps, and materials disclosed herein, as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior or invention.

It is noted that, as used in this specification and the appended claims, the singular forms “a,” “an,” and “the” include plural referents unless the context clearly dictates otherwise. Thus, for example, reference to a medical composition containing “a phytoestrogen” includes reference to a mixture of two or more of such phytoestrogens, reference to “an antioxidant” includes reference to one or more of such antioxidants, and reference to “a vitamin” includes reference to two or more of such vitamins.

In describing and claiming the preferred embodiments of the invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially effect the basic and novel characteristic or characteristics of the preferred embodiments.

Beneficial modulation of estrogen metabolism can be accomplished through dietary modification and supplementation with select nutrients. A weight management program can also be helpful in both reducing adipose aromatase activity and facilitating more desirable estrogen metabolism and excretion. The promotion of healthy estrogen metabolism in this way can have profound significance for diseases and conditions in which these hormones play a role.

Multiple dietary and nutritional factors can have the ability to influence estrogen synthesis and receptor activity, as well as the detoxification pathways through which estrogens are metabolized. Examples of interrelatedness of dietary and nutritional factors and estrogen synthesis and receptor activity are shown in Table 2. Incorporating dietary changes with the use of selected nutritional supplements can have profound effects in beneficially influencing estrogen balance and thus preventing estrogen-related diseases and conditions.

TABLE 2
Mechanisms through which dietary and nutritional
factors can influence estrogen metabolism

| Mechanism of Action | Nutrient |
|---|---|
| Promote C-2 hydroxylation over C-4 and/or | Cruciferous vegetables, indole-3-carbinol, |
| C-16α hydroxylation of estrogens | isoflavones (soy, kudzu) |
| Reduce the oxidation of catechol estrogens | Vitamins A, E, and C, N-acetylcysteine, |
| (2-OH and 4-OH) | turmeric, green tea, lycopene, α-lipoic acid, |
| | flavonoids |
| Promote the methylation of catechol | Folate, vitamins B2, B6, and B12, |
| estrogens (2-OH and 4-OH) | trimethylglycine, magnesium |
| Increase circulating concentrations of sex | Fiber, lignans (flaxseed), isoflavones (soy, |
| hormone binding globulin (SHBG), thus | kudzu) |
| reducing levels of unbound, active estrogens | |
| Inhibit the activity of aromatase, which | Lignans (flaxseed), flavonoids |
| converts testosterone and androstenedione | |
| into estradiol and estrone, respectively | |
| Promote the detoxification of estrogens by | Tumeric (curcumin), d-limonene, |
| upregulating Phase I and Phase II enzymes | magnesium, vitamins B2, B6, and B12, |
| | flavonoids |
| Inhibit the activity of β-glucoronidase, which | Fiber, probiotics (acidophilis, bifidobacteria), |
| deconjugates estrogens in the large intestine, | calcium D-glucarate |
| allowing them to be reabsorbed and re- | |
| metabolized | |
| Modify estrogen receptor activity | Isoflavones (soy, kudzu), lignans (flaxseed), |
| | indole-3-carbinol |

An article from Applied Nutritional Science Reports, 2001, pages 1-8, incorporated herein by reference, discloses nutritional influences on estrogen metabolism. The fact that PMS can be modified with hormone therapies suggests that endocrine metabolism can have a role in its etiology and/or symptoms. Data suggests low progesterone and/or excess estrogen levels, particularly during the early luteal phase, are observed in many women with PMS. A feature of PMS can be a relative imbalance in estrogen to progesterone activity. This imbalance can occur as increased levels of estrogen and/or changes in estrogen metabolism result in an increase in the highly estrogenic metabolites over that of the less active metabolite. The resulting relative estrogen dominance can account for some or all of the symptoms associated with PMS. By nutritionally modulating estrogen transport, metabolism, and excretion, it can be possible to improve some or all of the symptoms of PMS.

Perimenopause is the period immediately before the start of menopause and the first year after menopause and is characterized as a time of significant hormonal fluctuation. Aside from menstrual irregularity, perimenopause can lead to a variety of other signs and symptoms including, but not limited to, night sweats, hot flashes, vaginal dryness, headaches, and depression. Earlier theories on the etiology of perimenopausal symptoms, in particular vasomotor symptoms, focused on the notion that they were the result of low estrogen levels. However, recent evidence suggests that fluctuations in estrogen levels can create intermittent vasomotor symptoms. Accordingly, it has been set forth the premise that the perimenopause is a time of erratic estrogen production (both high and low), and that the times of spiking estrogen levels are causally connected with the clinical manifestations associated with this period. Overall, estrogen activity can be nutritionally supported with certain nutrients and dietary modifications. Nutritional interventions aimed at stabilizing or balancing these estrogen fluctuations can be safe, efficacious, and cost-effective alternative to hormone replacement therapy.

Preferred embodiments comprise a medical composition designed to nutritionally support mammals, particularly humans, with symptoms associated with their hormone cycles. Certain embodiments of the invention provide a combination of macronutrients and micronutrients to support healthy hormone cycles. Other embodiments of the invention can provide a combination of micronutrients, without macronutrients. A macronutrient is a nutrient that is needed in a large amount for growth and health of an animal; examples of macronutrients include, but not limited to, protein, lipids, and carbohydrates. A micronutrient is a nutrient that is needed in a small amount for growth and health of an animal.

Dietary Fiber and Lignan

Insoluble dietary fibers, such as lignan (found in flaxseeds and the bran layer of grains, beans, and seeds) can interrupt the enterohepatic circulation of estrogens in two ways, thus promoting their excretion and making them less available for reabsorption and further metabolism. First, dietary fiber, especially lignin, can bind to unconjugated estrogens in the digestive tract, which are then excreted in the feces. Second, dietary fiber can beneficially affect the composition of intestinal bacterial and reduce intestinal β-glucuronidase activity, resulting in a lowered deconjugation of estrogen and reduced reabsorption. Dietary fiber intake also increases serum concentrations of SHBG, thus reducing levels of free estradiol.

High-fiber, low-fat diets have been associated with lower levels of circulating estrogen in premenopausal women, as well as with a decreased risk of breast cancer. Certain types of fibers have been shown to preferentially bind steroids, in particular estrogen, suggesting that some fibers can preferentially decrease estrogen due to an increased absorptive capacity. Studies investigating the chemical nature of these fibers have shown that the component called lignan is responsible for the specificity of estrogen binding. Lignan is found at high levels in wheat and flax fibers.

Flaxseed meal is advantageously added to the medical composition of the preferred embodiments. Flaxseed meal contains lignin, which is the fiber that specifically binds hormones such as estrogen, thereby facilitating estrogen excretion. (C. J. M. Arts, Effects of Dietary Fiber on Breast Cancer Pathogenesis, in S. Gorog, Proc. Of the 5th Symp. On the Analysis of Steroids 575-585 (Szombathely, Hungary 1993); T. D. Shultz & J. B. Howie, In Vitro Binding of Steroid Hormones by Natural and Purified Fibers, 8 Nutr. Cancer 141-147 (1986)) Preferably, a medical composition of the preferred embodiments comprises about 0.1 to 20 parts by weight of defatted flaxseed meal, and more preferably about 0.5 to 10 parts by weight.

Carbohydrates

The medical composition of the preferred embodiments also comprises carbohydrates, as a macronutrient. Of the calorie sources, carbohydrates can be more readily utilizable than proteins or lipids to provide a source of energy for growth and maintenance of body tissue and to regulate body processes. The providing of energy is an important role of carbohydrates and can be satisfied at the expense of the other nutritive roles, if there are insufficient nutrients to accomplish these functions. Carbohydrates are made up of simple sugars or monosaccharides, oligosaccharides (such as di- and tri-saccharides), and polysaccharides.

Of the simple sugars, hexoses (glucose and fructose, in particular) are important to energy production and to regulating body processes. When simple sugars are metabolized, energy is released. However, to be utilized as a source of energy in the body, carbohydrates are first degraded into simple sugars. Metabolic processes convert the simple sugars into various products, such as carbon dioxide and water, or alcohols and, in the case of fermentation in muscular tissues, to lactic acid, accompanied by the release of energy. About 20 percent of simple sugar metabolism can give rise to lactic acid production. These simple sugars or monosaccharides are also utilized as raw materials for synthesis of a variety of organic compounds, such as steroids, amino acids, purines, pyrimidines, complex lipids, and polysaccharides and the like.

Of the various simple sugars, glucose is prevalent as a base source of energy. However, glucose stimulates the production of insulin, which is used for proper glucose metabolism. Fructose, on the other hand, does not require insulin to enter certain cells of the body and therefore results in a smooth indirect flow into the bloodstream and from there, to the brain and other portions of the body. Moreover, fructose can also promote a more rapid emptying of the stomach. In not delaying gastric emptying, there is a reduced feeling of bloating and also a more rapid delivery of the nutrients into the small intestine for uptake into the portal blood. Both glucose and fructose can be readily assimilated and metabolized.

Because of the relative ease with which fructose is assimilated, coupled with the fact that it does not require insulin for metabolism, fructose is preferably used in the preferred embodiments. Fructose, or fruit sugar, can be obtained from fruit sources or from the hydrolysis of sucrose. Sucrose, or table sugar, is a disaccharide made up of glucose and fructose and, upon hydrolysis, yields one molecule of each simple sugar.

Accordingly, sources of carbohydrates that can be used in the preferred embodiments include fructose and rice syrup solids. In addition, it has been found to be beneficial to add xylitol and alpha-D-ribofuranose to the medical composition of the preferred embodiments.

Fats and Fat Modulators

Balance among types and amounts of dietary fats can play a role in determining balance among estrogens in the body. In male chimpanzees fed a high-fat, low-carbohydrate, low-protein diet for eight weeks, estradiol was metabolized primarily through C-16α hydroxylation, whereas it was metabolized primarily through C-2 hydroxylation in chimpanzees fed a normal diet. Breast cancer cells exposed to eicosapentaenoic acid, an omega-3 fatty acid found in cold-water fish, showed increases in C-2 hydroxylation of estradiol and decreases in C-16α hydroxylation of estradiol. Women with severe premenstrual breast symptomology who reduced their intake of fat while increasing their consumption of complex carbohydrates experienced significant symptom reduction.

The medical composition of the preferred embodiments also comprises a source of dietary fat, as a macronutrient. Preferably, this dietary fat comprises canola oil that is high in oleic acid, choline, and the like and mixtures thereof. Choline helps a body absorb and use fats. Choline also aids in methylation reactions that occur in the body. Preferably, the medical composition comprises about 0.01 to 10 parts by weight of fat, and more preferably about 0.1 to 6 parts by weight. Preferably, the preferred embodiments comprise about 0.1×10⁻³ to 750×10⁻³ parts by weight of choline, and more preferably about 1×10⁻³ to 500×10⁻³ parts by weight.

Protein

Inadequate dietary protein can lead to decreases in overall cytochrome P450 activity, including cytochrome P450-1A2, which detoxifies estradiol. Rice is source of protein frequently used to nutritionally support hepatic detoxification function, because of its low allergy potential. Additionally, fortifying rice protein with lysine and threonine resulted in better support of hepatic mitochondrial functions in rats fed a rice protein-based diet as compared to rats fed a casein protein-based diet or a rice-protein-based diet without lysine and threonine supplementation.

A source of protein as a macronutrient in the present medical composition is a low-allergy-potential rice protein concentrate, as disclosed in U.S. Pat. No. 4,876,096 and incorporated herein by reference. This rice protein concentrate provides a complete, high-quality, easily digestible vegetable protein. The preferred embodiments also preferably include rice flour as an additional source of vegetable protein.

Phytoestrogens

Phytoestrogens are plant estrogens that have the capacity to bind to ERs and have both estrogenic and anti-estrogenic effects, depending on the expression of ER subtypes in target cells and on the level of endogenous estrogen present. Phytoestrogens are currently being extensively investigated as a potential alternative for a range of conditions associated with estrogen imbalance, including, but not limited to, menopausal symptoms, premenstrual syndrome, endometriosis, prevention of breast and prostate cancer, and protection against cardiovascular disease and osteoporosis. The two main classes of phytoestrogens are isoflavones and lignans.

Many of the benefits of increased intakes of dietary phytoestrogens are due to their ability to beneficially influence estrogen synthesis and metabolism through a variety of mechanisms: 1) they have a similar structure to estradiol and can bind to the ER, 2) they increase plasma levels, 3) they decrease aromatase activity, and 4) they shift estrogen metabolism away from the C-16α pathway to the C-2 pathway.

Flavonoids

Flavonoids (also called bioflavonoids) are natural botanical pigments that provide protection from free-radical damage, among other functions. Bioflavonoids can provide protection from damaging free radicals and are believed to reduce the risk of cancer and heart disease, decrease allergy and arthritis symptoms, promote vitamin C activity, improve the strength of blood vessels, block the progression of cataracts and macular degeneration, treat menopausal hot flashes, and other ailments. Flavonoids occur in most fruits and vegetables. It is believed that flavonoids act by inhibiting hormones, such as estrogen, that can trigger hormone-dependent malignancies, like cancers of the breast, endometrium, ovary, and prostate. Studies show that quercetin, a flavonoid found in citrus fruits, can block the spread of cancer cells in the stomach. Flavonoids can also stabilize mast cells, a type of immune cell that releases inflammatory compounds, like histamine, when facing foreign microorganisms. Histamine and other inflammatory substances are involved in allergic reactions. Mast cells are large cells present in connective tissue. Flavonoids fortify and repair connective tissue by promoting the synthesis of collagen. Collagen is a remarkably strong protein of the connective tissue that “glues” the cells together. Flavonoids are believed to benefit connective tissue and reduce inflammation. Chrysin is a flavone that can be added to a medical composition of the preferred embodiments.

Hesperidin complex is a bioflavonoid that can be also advantageously added to the medical composition of the preferred embodiments. Hesperidin can be found in the rinds of oranges and lemons. It can help strengthen papillary walls in conjunction with vitamin C.

Isoflavones

Isoflavones are a group of phytochemicals that can provide beneficial effects when provided as supplements to the diet. Isoflavones are phytoestrogens that are about one-hundredth to one-thousandth as potent as human estrogen. Isoflavones can bind to the estrogen receptor and, therefore, compete with, or block, estrogen actions. Furthermore, isoflavones can serve in some cases as antagonists to estrogen binding and in others as agonists. In this way, isoflavones can be considered hormonal adaptogens. Although they are weak estrogens, isoflavones can help offset the drop in estrogen that occurs naturally at menopause. Isoflavones can act like hormone replacement therapy (HRT), easing hot flashes in menopausal women.

Isoflavones can also increase hepatic SHBG synthesis, which, in theory, lowers risk of hormone-related cancers by decreasing the amount of free or active hormone present in the blood. Higher intakes of soy products and other isoflavones, such as consumed in traditional Japanese diets, are associated with low rates of hormone-dependent cancers. The average daily isoflavone intake of Japanese women is about 20 to 80 mg, while that of American women is about 1 to 3 mg. Additionally, women given about 45 mg of isoflavones daily for one month experienced longer menstrual cycles (increased number of days between menstruation) and lower luteinizing hormone and follicle-stimulating hormone surges. Young women consuming about 36 ounces of soymilk daily for one month also experienced longer menstrual cycles (about 28.3±1.9 days before soymilk feeding) and lower serum estradiol levels, both effects which persisted for two to six menstrual cycles after discontinuation of the soymilk. In women with low levels of SHBG, consumption of a soymilk powder providing about 69 mg of isoflavones daily substantially increased their SHBG concentrates, an effect not observed in women with higher initial SHBG levels.

Isoflavones and soy protein also can prevent bone loss that leads to osteoporosis. Also, soy protein is being investigated for its lipid lowering effects.

The most researched isoflavones are genistein, daidzein and glycitein. Data on the isoflavone content of foods is limited; however, the United States Department of Agriculture (USDA)—Iowa State University Isoflavone Database lists some common foods and their isoflavone content. Kudzu root is high in isoflavones, such as daidzein and genistein, and isoflavone glycosides, such as daidzin and puerarin. (P. B. Kaufman et al., A Comparative Survey of Leguminous Plants as Sources of the Isoflavones Genistein and Daidzein: Implications for Human Nutrition and Health, 3 J. Altern. Complement Med. 7-12 (1997)) These isoflavones and/or their metabolites bind to the estrogen receptor and act as weaker estrogens, resulting in an inhibition of the estrogenic effect. (G. G. J. M. Kuiper et al., Interaction of Estrogenic Chemicals and Phytoestrogens with Estrogen Receptor β, 139 Endocrinology 4252-4263 (1998); A. Cassidy, Potential Tissue Selectivity of Dietary Phytoestrogens and Estrogens, 10 Curr. Opin. Lipdol. 47-52 (1999); S. R. Davis et al., Phytoestrogens in Health and Disease, 54 Recent Prog. Horm. Res. 185-210 (1999); M. E. Martin et al., Interactions between Phytoestrogens and Human Sex Steroids Binding Protein, 58 Life Sci. 429-436 (1996))

The main dietary sources of isoflavones are in foods such as, but not limited to, kudzu root, soy, legumes, alfalfa, clover, and licorice root. It is not clear the amount of soy that is needed to get the most health benefit. Studies have shown that it can take as little as about 20 grams of soy protein (about half an ounce), or about 2 cups of soy milk, or about 2 ounces of tofu daily to help ease symptoms.

Certain embodiments comprise about 0.1×10⁻³ to 500×10⁻³ parts by weight, preferably about 1×10⁻³ to 50×10⁻³ parts by weight, and more preferably about 10×10⁻³ to 40×10⁻³ parts by weight of isoflavones from kudzu. Other embodiments comprise about 0.2×10⁻³ to 1000×10⁻³ parts by weight, preferably about 2×10⁻³ to 100×10⁻³ parts by weight, and more preferably about 20×10⁻³ to 80×10⁻³ parts by weight of isoflavones from kudzu.

Lignans

These compounds are found in fiber-rich foods and, through intestinal fermentation, are converted into mammalian lignans with greater biological activity, such as enterolactone and enterodiol. Lignans stimulate the production of SHBG in the liver, and therefore reduce the levels of free estrogen in circulation. Enterolactone inhibits aromatase activity, and may thereby decrease the conversion of testosterone and androstenedione into estrogens in fat and breast cells. Lignans also have been shown to inhibit estrogen-sensitive breast cancer cell proliferation. Women consuming about 10 grams of flaxseed, which contains lignans, per day experienced longer menstrual cycle length, increased progesterone-to-estrogen ratios, and fewer anovulatory cycles, all of which were considered to reflect improved ovarian function. Through their detrimental effects on intestinal flora, antibiotics may reduce the formation of mammalian lignans.

Isoflavone Synergists

Various ingredients have been shown to have a synergistic beneficial effect on the health of the hormonal cycle in the presence of isoflavones. Curcumin, an active component in turmeric (Curcuma longa), combined with dietary isoflavones gives in vitro evidence of reducing xenoestrogen-induced growth in estrogen receptor-positive and -negative cells. (S. P. Verma et al., Curcumin and Genistein, Plant Natural Products, Show Synergistic Inhibitory Effects on the Growth of Human Breast Cancer MCF-7 Cells Induced by Estrogenic Pesticides, 233 Biochem. Biophys. Res. Comm. 692-696 (1997)) Curcumin has also been shown to play a role in detoxification through its ability to induce glutathione production and glutathione-S-transferase activity. (M. Susan & M. N. A. Rao, Induction of glutathione-S-transferase Activity by Curcumin in Mice, 42 Drug Res. 962-964 (1992))

Curcumin has long been recognized for pharmacological properties, such as anti-inflammatory, anti-tumor, and antioxidant. However, the combination of curcumin and isoflavones produce a more potent effect than the individual compounds in of reducing xenoestrogen-induced growth in estrogen receptor-positive and -negative cells. Curcumin can provide a synergistic effect by acting on the same or different pathways as those of the isoflavones. Curcumin can act on enzymes involved in growth signaling. Curcumin can also suppress the activities of protein kinases and many types of transcription factors and proto-oncogenes.

Certain embodiments comprise about 1×10⁻³ to 5000×10⁻³ parts by weight, preferably about 50×10⁻³ to 500×10⁻³ parts by weight, and more preferably about 100×10⁻³ to 300×10⁻³ parts by weight of curcumin. Other embodiments comprise about 0.5×10⁻³ to 2500×10⁻³ parts by weight, preferably about 25×10⁻³ to 250×10⁻³ parts by weight, and more preferably about 50×10⁻³ to 150×10⁻³ parts by weight of curcumin.

Other compounds that can be synergistic with isoflavones are, but not limited to, resveratrol and rosemary extract. Certain embodiments comprise about 0.1×10⁻³ to 100×10⁻³ parts by weight, preferably about 0.5×10⁻³ to 50×10⁻³ parts by weight, more preferable about 0.5×10⁻³ to 10×10⁻³ parts by weight of resveratrol. Certain embodiments comprise about 1×10⁻³ to 1000×10⁻³ parts by weight, preferably about 10×10⁻³ to 500×10⁻³ parts by weight, more preferable about 25×10⁻³ to 200×10⁻³ parts by weight of rosemary extract.

Methylation Support Compounds

Estrogenic hormones are detoxified and eliminated from the body by conversion to hormonally inactive water-soluble metabolites. The detoxification process starts by way of Phase I cytochrome P-450 activation (i.e., mono-oxidation or hydroxylation), followed by Phase II glucoronidation, sulfation, and/or O-methylation. The Phase I pathway serves to biotransform substances through oxidation, reduction or hydrolysis, using the cytochrome P450 oxidase enzymes. Once the substance is transformed, the substance has increased solubility and is subsequently prepared for the Phase II pathway. The Phase II pathway for metabolism of estrogen include methylation, glucoronidation, and sulfation. Enzymes in the Phase II pathway include methyltransferases, sulfotranferases, and glucuronyl transferase.

It is preferably to detoxify estrogenic hormones to the Phase II stage. O-methylated derivatives of 2-hydroxyestradiol have been found to be potent inhibitors of tumor cell proliferation and angiogenesis. On the other hand, C-16α-hydroxylated estrogens are active estrogens and induce mammary tumors in animals. Hence, it is favorable to methylate the C-16α-hydroxylated estrogens to aid in detoxification and elimination from the body. Examples of Phase II enzymes that perform methylation include COMT and S-adenosyl-L-methionine:delta-24 [25]sterol methyltransferase.

The O-methylation of estrogenic metabolites is catalyzed by the COMT and uses SAM as a methyl donor. Therefore, the co-factors used for methylation support, such as methylfolate, cobalamin, and pyrodixine, help support this pathway. Other compounds used for methylation support include choline, trimethylglycine, riboflavin, and magnesium.

Vitamins

Vitamins are organic compounds that are used for the normal growth and maintenance of life of animals, including man, who are generally unable to synthesize these compounds by anabolic processes that are independent of environment other than air. Vitamins are effective in small amounts, do not furnish energy, and are not utilized as building units for the structure of the organism, but are essential for the transformation of energy and for the regulation of the metabolism of structural units. Vitamins or their precursors are found in plants, and thus plant tissues are the sources for the animal kingdom of these protective nutritional factors. The food of humans and animals should contain small amounts of vitamins to promote good health of man and animals. Thirteen well-defined vitamins include vitamin A, vitamin D, vitamin E, vitamin K, eight B vitamins (vitamin B-1, vitamin B-2, vitamin B-3, vitamin B-6, vitamin B-12, folic acid, pantothenic acid, and biotin), and vitamin C. If any one of at least thirteen of these compounds is lacking in the diet, a breakdown of the normal metabolic processes can occur, which results in a reduced rate or complete lack of growth in children and in symptoms of malnutrition that are classified as deficiency diseases.

Functions of vitamins generally fall into two categories, the maintenance of normal structure and the maintenance of normal metabolic functions. For example, vitamin A is used for the maintenance of normal epithelial tissue, and vitamin D functions in the absorption of normal bone salts for the formation and growth of a sound bone structure. Certain vitamins, such as thiamine, riboflavin, pantothenic acid, and niacin, are known to be constituents of the respiratory enzymes that are used in the utilization of energy from oxidative catabolism of sugars and fats.

It is convenient to divide vitamins into two groups, water-soluble vitamins and fat-soluble vitamins. The water-soluble vitamins include vitamin C and the B group of vitamins. The fat-soluble vitamins include vitamins A, D, E, and K, since they can be extracted with organic solvents and are found in the fat fractions of animal tissues. For brief reviews of vitamins in general and specific vitamins, see Remington's Pharmaceutical Sciences.

Fat Soluble Vitamins

Vitamin A is used for the maintenance of normal tissue structure and for other physiological functions, such as vision and reproduction. A source of vitamin A in animals is the carotenoid pigments, i.e. the yellow-colored compounds in chlorophyll-containing plants. At least 10 different carotenoids exhibit provitamin A activity. For example, α- and β-carotene and cryptoxanthin (found in yellow corn) are important in animal nutrition, while β-carotene being more important. Theoretically, one molecule of β-carotene can yield two molecules of vitamin A. The availability of carotene in foods as sources of vitamin A for humans, however, is low and variable. The conversion of the provitamin to vitamin A occurs primarily in the walls of the small intestine and perhaps to a lesser degree in the liver. Like vitamin A, the carotenes are soluble in organic solvents.

Of the known functions of vitamin A in the body, its role in vision is well-established. The retina of man contains two distinct photoreceptor systems. The rods, which are the structural components of one system, are especially sensitive to light of low intensity. A specific vitamin A aldehyde is used for the formation of rhodopsin, the high molecular weight glycoprotein part of the visual pigment within the rods, and the normal functioning of the retina. By virtue of this relation in the visual process, vitamin A alcohol has been named retinol, and the aldehyde form is named retinal. A vitamin-A deficient person has impaired dark adaption (“night-blindness”).

Vitamin A also aids in the differentiation of cells of the skin (lining the outside of the body) and mucous membranes (linings inside of the body); helps the body fight off infection and sustain the immune system; and, supports growth and remodeling of bone and teeth. In addition, dietary vitamin A, in the form of its precursor β-carotene (an antioxidant), can help reduce risk for certain cancers. In the preferred embodiments, vitamin A is preferably supplied as retinyl palmitate.

Vitamin D is effective in promoting calcification of the bony structures of man and animals. It is sometimes known as the “sunshine” vitamin because it is formed by the action of the sun's ultraviolet rays on precursor sterols in the skin. Vitamin D aids in the absorption of calcium from the intestinal tract and the resorption of phosphate in the renal tubule. Vitamin D is utilized for normal growth in children, probably having a direct effect on the osteoblast cells, which influence calcification of cartilage in the growing areas of the bone. A deficiency of vitamin D can lead to inadequate absorption of calcium from the intestinal tract and retention of phosphorus in the kidney and thus, to faulty mineralization of bony structures. Vitamin D also helps to maintain a stable nervous system and normal heart action.

Vitamin E is a group of compounds (tocol and tocotrienol derivatives) that exhibit qualitatively the biological activity of α-tocopherol. Biological activity associated with the vitamin nature of the group is exhibited by four major compounds: α-, 62 -, γ-, and δ-tocopherol, each of which can exist in various stereoisomeric forms. The tocopherols act as antioxidants, while δ-tocopherol having the greatest antioxidant power. A certain function of vitamin E occurs in the membranous parts of the cells. Vitamin E interdigitates with phospholipids, cholesterol, and triglycerides, which are the three main structural elements of the membranes. Since vitamin E is an antioxidant, a favored reaction occurs with very reactive and highly destructive compounds called free radicals. Free radicals are products of oxidative deterioration of such substances as polyunsaturated fat. Vitamin E converts the free radical into a less reactive and a nonharmful form. Vitamin E can also help supply oxygen to the blood, which is then carried to the heart and other organs; thus alleviating fatigue. Vitamin E can also aid in bringing nourishment to cells; strengthen the capillary walls and prevent the red blood cells from destructive poisons; prevent and dissolve blood clots; and be used in helping prevent sterility, muscular dystrophy, calcium deposits in blood walls, and heart conditions. In the preferred embodiments, vitamin E is preferably supplied in the form of d-alpha-tocopheryl succinate. Vitamin E can aid in managing symptoms of PMS.

Vitamin K is involved in the blood-clotting system through synthesis of prothrombin and other clotting factors. Vitamin K can be used for the formation of prothrombinogen and other blood clotting factors in the liver. During clotting, circulating prothrombin is used for the production of thrombin. In turn, thrombin converts fibrinogen to fibrin, the network of which constitutes the clot. Interference with formation of prothrombin can reduce clotting tendency of blood. In a deficiency of vitamin K, a condition of hypoprothrombinemia can occur, and blood-clotting time can be greatly, or even indefinitely, prolonged. Internal or external hemorrhages can ensue either spontaneously or following injury or surgery.

Water-Soluble Vitamins

Except for vitamin C (ascorbic acid), the vitamins in this category belong the B-group of vitamins. Some still retain their original individual designations, such as B-1, B-6, and B-12, whereas comparable names for other vitamins have become obsolete.

Vitamin C, or ascorbic acid, is known to be used for the formation of intercellular collagen. Symptoms of scurvy, due to vitamin C deficiency, include bleeding gums, easy bruising and a tendency toward bone fractures. These symptoms are a result of discrepancies in the development of the ground substance between our cells, a role of vitamin C. The ground substance, primarily collagen, is the cement that gives tissues form and substance. Collagens are components of tendons, ligaments, skin, bone, teeth, cartilage, heart valves, intervertebral discs, cornea, eye lens, in addition to the ground substance between cells. Collagen can form in the absence of ascorbic acid, but the fibers formed from the absence of ascorbic acid are abnormal, resulting in skin lesions and blood vessel fragility, which are characteristics of scurvy. In scorbutic tissues, the amorphous ground substance and the fibroblasts in the area between the cells appear normal, but the tissue lacks the matrix of collagen fibers. In tissues that lack the matrix of collagen fibers, bundles of collagenous material can appear within a few hours after administration of ascorbic acid. This effect points to the relationship of vitamin C to the maintenance of tooth structures, matrix of bone, and the walls of capillaries. Vitamin C is also used for the healing of bone fractures. Such fractures can heal slowly in a patient deficient in vitamin C. This result is true also of wound healing.

Vitamin C is also an antioxidant. Oxygen is a highly reactive element, and the process of reacting with certain chemicals is termed oxidation. Oxidation is not always bad. For example, the iron in hemoglobin oxidizes to carry oxygen to all the cells of the body. But most oxidation is damaging, resulting in accelerating aging and contributing to tissue and organ damage. Oxidation is also a contributor to heart disease low density lipoprotein (LDL) oxidation has been linked to atherosclerosis and cancer. As research continues, free-radical damage appears to contribute to chronic conditions and antioxidant nutrition supplementation is realized to be is useful to good health. Vitamin C is an effective water-soluble antioxidant in human plasma. Vitamin C is also used for the proper functioning of the immune system. It is involved in white blood cell production, T-cells, and macrophages. In the preferred embodiments, vitamin C is preferably supplied in forms, such as, but not limited to, calcium ascorbate, niacinamide ascorbate, L-xyloascorbic acid, sodium ascorbate, magnesium ascorbate, ascorbyl palmitate, and potassium ascorbate, and mixtures thereof.

Biotin (Vitamin B7) functions in synthesis and breakdown of fatty acids and amino acids through aiding the addition and removal of carbon dioxide to or from active compounds. It similarly acts in catalyzing deamination of amino acids and in oleic acid synthesis. Biotin is also a component of enzymes and aids in the utilization of protein and certain other vitamins, such as folic acid, pantothenic acid, and vitamin B-12.

Folic acid (Vitamin B9 or folacin) and derivatives thereof are important hematopoietic agents used for proper regeneration of blood-forming elements and their functioning. 5-methyltetrahydrofolate is a derivative of folic acid. Folic acid is involved as a coenzyme in intermediary metabolic reactions in which one-carbon units are transferred. Accordingly, folic acid and derivatives thereof are can aid in methylation of estrogenic compounds. These methylation reactions are also utilized in interconversions of various amino acids and in purine and pyrimidine synthesis. The biosynthesis of purines and pyrimidines is ultimately linked with that of nucleotides and ribo- and deoxyribo-nucleic acids, which are functional elements in all cells.

Niacin (nicotinic acid) (Vitamin B3) and niacinamide (nicotinamide) have substantially the same properties, as vitamins. In the body, niacin is converted to niacinamide, which is a constituent of coenzymes I and II that is used in a wide variety of enzyme systems involved in anaerobic oxidation of carbohydrates. The coenzyme serves as a hydrogen acceptor in the oxidation of the substrate. These enzymes are present in living cells and take part in many reactions of biological oxidation. Nicotinamide-adenine dinucleotide (NAD) and nicotinamide-adenine dinucleotide phosphate (NADP) are coenzymes synthesized in the body that take part in the metabolism of living cells. Since they are of such widespread and vital importance, disturbance of metabolic processes can occur when the supply of niacin to the cell is interrupted. Niacin is readily absorbed from the intestinal tract, and large doses can be given orally or parenterally with equal effect. Further, niacin can improve circulation and reduce cholesterol level in the blood; maintain the nervous system; help metabolize protein, sugar and fat; reduce high blood pressure; increase energy through proper utilization of food; prevent pellagra; and help maintain a healthy skin, tongue, and digestive system. In the preferred embodiments, niacin is preferably provided as, but not limited to, niacin, niacinamide, niacinamide ascorbate, and the like, and mixtures thereof.

Pantothenic acid (Vitamin B5) is of biological importance because of its incorporation into Coenzyme A (CoA), which is involved in many vital enzymatic reactions transferring a two-carbon compound (the acetyl group) in intermediary metabolism. It is involved in the release of energy from carbohydrate and protein, in the degradation and metabolism of fatty acids, and in the synthesis of such compounds as sterols and steroid hormones, porphyrins, acetyl-choline, and the like. Pantothenic acid can also participate in the utilization of vitamins; improve the body's resistance to stress; help in cell building and the development of the central nervous system; help the adrenal glands; and fight infections by participating in building of antibodies. In the preferred embodiments, pantothenic acid is preferably provided in the form of the acid, salts thereof, or mixtures thereof. A preferred salt of pantothenic acid is d-calcium pantothenate.

Pyridoxine (vitamin B-6) does not denote a single substance, but is rather a collective term for a group of naturally occurring pyridines that are metabolically and functionally interrelated: namely, pyridoxine, pyridoxal, and pyridoxamine. They are interconvertible in vivo in their phosphorylated form. Vitamin B-6 in the form of pyridoxal phosphate or pyridoxamine phosphate functions in carbohydrate, fat, and protein metabolism. Its major functions are most closely related to protein and amino acid metabolism. Pyridoxine is a part of the molecular configuration of many enzymes (a coenzyme), notably glycogen phosphorylase, various transaminases, decarboxylases, and deaminases. The latter three are used for the anabolism and catabolism of proteins. Pyridoxine is also aids in fat and carbohydrate metabolism; aids in the formation of antibodies; maintains the central nervous system; aids in the removal of excess fluid of premenstrual women; promotes healthy skin; reduces muscle spasms, leg cramps, hand numbness, nausea and stiffness of hands; and helps maintain a proper balance of sodium and phosphorous in the body. In the preferred embodiments, pyridoxine is preferably provided in the acid addition salt form as pyridoxine hydrochloride.

Pyridoxine aids as a methylation support compound by providing help in synthesizing SAM. Also, pyridoxine modulates the ability of cells in vitro to respond to steroid hormones. Low levels of pyridoxine in the system can lead to prolonged and increased estrogenic response, whereas high levels of pyridoxine have shown an attenuated estrogenic response in cell culture studies. (D. B. Tully et al., Modulation of Steroid Receptor-mediated Gene Expression by Vitamin B6, 8 FASEB J. 343-349 (1994)) Studies regarding discomfort during hormone cycles suggest that women's intake ratio between pyridoxine and protein should be greater than about 0.016 mg/g. (D. A. Bender, Novel Functions of Vitamin B6, 3 Proc. Nutr. Soc. 625-630 (1994); C. M. Hansen et al., Changes in Vitamin B-6 Status indicators of Women Fed a Constant Protein Diet with Varying Levels of Vitamin B-6, 66 Am. J. Clin. Nutr. 1379-1387 (1997)) The preferred embodiments preferably surpasses this ratio, with a pyridoxine/protein ratio of about 2 mg/g, more preferably about 1 mg/g, even more preferably about 0.727 mg/g. Some studies have shown that pyridoxine decreases premenstrual symptoms and depression at doses of up to about 100 mg per day. (K. M. Wyatt et al., Efficacy of Vitamin B-6 in the Treatment of Premenstrual Syndrome: Systematic Review, 318 BMJ 1375-1381 (1999); M. K. Berman et al., Vitamin B-6 in Premenstrual Syndrome, 90 Am. J. Diet. Assoc. 859-861 (1990); M. C. DeSouza et al., A Synergistic Effect of a Daily Supplement for 1 month of 200 mg Magnesium plus 50 mg Vitamin B6 for the Relief of Anxiety-related Premenstrual Symptoms: A Randomized, Double-blind, Crossover Study, 9 J. Womens Health Gend. Based Med. 131-139 (2000))

Riboflavin (Vitamin B2) plays a physiological role as the prosthetic group of a number of enzyme systems that are involved in the oxidation of carbohydrates and amino acids. It aids in the methylation support of estrogenic metabolites. Also, it functions in combination with a specific protein either as a mononucleotide containing phosphoric acid (FMN), or as a dinucleotide combined through phosphoric acid with adenine (FAD). The specificity of each of the enzymes is determined by the protein in the complex. By a process of oxidation-reduction, riboflavin in the system either gains or loses hydrogen. The substrate, either carbohydrate or amino acid, can be oxidized by a removal of hydrogen. The first hydrogen acceptor in the chain of events is NAD or NADP, the di- or tri-nucleotide containing nicotinic acid and adenine. The oxidized riboflavin system then serves as hydrogen acceptor for the coenzyme system and in turn is oxidized by the cytochrome system. The hydrogen is finally passed on to the oxygen to complete the oxidative cycle. A number of flavoprotein enzymes have been identified, each of which is specific for a given substrate. Riboflavin also aids in the formation of antibodies and red blood cells; maintains cell respiration; is used for the maintenance of good vision, skin, nails and hair; alleviates eye fatigue; and promotes general good health.

Thiamine (Vitamin B1) is a generic term applied to substances possessing vitamin B-1 activity, regardless of the anion attached to the molecule. The cationic portion of the molecule is made up of a substituted pyrimidine ring connected by a methylene bridge to the nitrogen of a substituted thiazole ring. In a phosphorylated form, thiamine serves as the prosthetic group of enzyme systems that are concerned with the decarboxylation of α-ketoacids. Some decarboxylation reactions are reversible, so that synthesis (condensation) may be achieved. Thus, thiamine is also important to the biosynthesis of keto-acids. It is involved in transketolase reactions. Thiamine is readily absorbed in aqueous solution from both the small and large intestine, and is then carried to the liver by the portal circulation. In the liver, as well as in all living cells, it normally combines with phosphate to form cocarboxylase. It can be stored in the liver in this form or it can be combined further with manganese and specific proteins to become active enzymes known as carboxylases. Thiamine also plays a role in the body's metabolic cycle for generating energy; aids in the digestion of carbohydrates; is used for the normal functioning of the nervous system, muscles and heart; stabilizes the appetite; and promotes growth and good muscle tone. In the preferred embodiments, thiamine is preferably provided in the acid addition salt form as thiamine hydrochloride.

Cobalamin (Vitamin B-12) and derivatives thereof are used for the functioning of cells, but particularly for cells of the bone marrow, the nervous system, and the gastrointestinal tract. Methylcobalamin and cyanocobalamin are derivatives of cobalamin. It appears to facilitate reduction reactions and participate in the transfer of methyl groups. Accordingly, cobalamin and derivatives thereof are can aid in methylation of estrogenic metabolites. A role of cobalamin seems to be also, together with folic acid, in the anabolism of DNA in cells. It is used for normal blood formation; and certain macrocystic anemias respond to its administration. Vitamin B-12 is also used for carbohydrate, fat, and protein metabolism; maintains a healthy nervous system; promotes growth in children; increases energy; and is used for calcium absorption.

Cobalamin, folic acid, pyridoxine, and riboflavin provide support for methylation pathways, such as homocysteine metabolism and methylation of estrogens. Methylenetetrahydrofolate reductase (MTHFR) is the enzyme responsible for providing methylated folate, which is a way a cell transfers methyl groups from one place to another. Plasma levels of methylated folate are decreased in individuals with a particular polymorphism in the MTHFR gene, which is common in the North American population. Bioavailable dietary supplies of folic acid and cobalamin can be used to adequately support MTHFR, and may be particularly helpful in individuals with this polymorphism.

Preferred formulations and ranges of these ingredients in the preferred embodiments are shown in Table 3 below.

TABLE 3
Preferred Formulations and Ranges of Vitamins

| | Ranges in Parts by Weight of | | | |
|---|---|---|---|---|
| | International Units (IU) | | | |
| Vitamins | Preferred | | More Preferred | |
| A | 50-20,000 | IU | 200-15,000 | IU |
| D | 25-1,000 | IU | 50-800 | IU |
| E | 25-800 | IU | 50-700 | IU |
| K | 1-400 × 10⁻⁶ | | 5-300 × 10⁻⁶ | |
| C | 1-5,000 × 10⁻³ | | 10-3,000 × 10⁻³ | |
| Thiamine (B1) | 50-5000 × 10⁻⁶ | | 100-2000 × 10⁻⁶ | |
| Riboflavin (B2) | 50-5000 × 10⁻⁶ | | 100-2000 × 10⁻⁶ | |
| Niacin (B3) | 0.5-50 × 10⁻³ | | 5-50 × 10⁻³ | |
| Pantothenic Acid (B5) | 0.1-200 × 10⁻³ | | 1-100 × 10⁻³ | |
| Pyridoxine (B6) | 0.1-500 × 10⁻³ | | 1-250 × 10⁻³ | |
| Folate (B9) | 50-5000 × 10⁻⁶ | | 100-1,000 × 10⁻⁶ | |
| Cobalamin (B12) | 2-200 × 10⁻⁶ | | 5-100 × 10⁻⁶ | |
| Biotin (B7) | 10-5,000 × 10⁻⁶ | | 50-1,000 × 10⁻⁶ | |

Minerals

Minerals can serve a wide variety of physiological functions ranging from structural components of body tissues to components of many enzymes and other biological important molecules. Minerals are classified as micronutrients or trace elements on the basis of the amount present in the body. The seven micronutrients (calcium, potassium, sodium, magnesium, phosphorus, sulfur, and chloride) are present in the body in quantities of more than about five grams. Trace elements, which include boron, copper, iron, manganese, selenium, and zinc are found in the body in quantities of less than about five grams.

Micronutrient Minerals

Calcium is the mineral element believed to be most deficient in the diet in the United States. Calcium intakes in excess of about 300 mg per day are difficult to achieve in the absence of milk and dairy products in the diet. This is far below the recommended dietary allowance (RDA) for calcium (about 1000 mg per day for adults and children ages one to ten, about 1200 mg per day for adolescents and pregnant and lactating women, which equates to about four glasses of milk per day). In fact, it has been reported that the mean daily calcium intake for females over age 12 does not exceed about 85 percent of the RDA. In addition, during the years of peak bone mass development (ages 18 to 30), more than about 66 percent of all U.S. women fail to consume the recommended amounts of calcium on any given day. After age 35, this percentage increases to over about 75 percent.

Although the general public is not fully aware of the consequences of inadequate mineral intake over prolonged periods of time, there is considerable scientific evidence that low calcium intake is one of several contributing factors leading to osteoporosis. In addition, the dietary ratio of calcium to phosphorous (Ca:P) relates directly to bone health. A Ca to P ratio of 1:1 to 2:1 is recommended to enhance bone marrowization in humans. Such ratios are difficult to achieve absent an adequate dietary supply of milk and dairy products, or an adequate supply of calcium and other minerals for the lactose-intolerant segment of the population. Additionally, calcium can help manage symptoms of PMS.

In the preferred embodiments, calcium can be added as inorganic, organic, or chelated form, or mixtures thereof. A preferred form of calcium comprises calcium citrate.

Magnesium is the second most plentiful cation of the intracellular fluids. It is used for the activity of many enzyme systems and plays a role with regard to neurochemical transmission and muscular excitability. Deficits are accompanied by a variety of structural and functional disturbances. The average 70-kg adult has about 2000 mEq of magnesium in his body. About 50% of this magnesium is found in bone, about 45% exists as an intracellular cation, and about 5% is in the extracellular fluid. About 30% of the magnesium in the skeleton represents an exchangeable pool present either within the hydration shell or on the crystal surface. Mobilization of the cation from this pool in bone is fairly rapid in children, but not in adults. The larger fraction of magnesium in bone is apparently an integral part of bone crystal.

The average adult in the United States ingests about 20 to 40 mEq of magnesium per day in an ordinary diet, and of this, about one third is absorbed from the gastrointestinal tract. The evidence suggests that the bulk of the absorption occur in the upper small bowel. Absorption is by means of an active process apparently closely related to the transport system for calcium. Ingestion of low amounts of magnesium results in increased absorption of calcium and vice versa.

Magnesium is a cofactor of enzymes involved in phosphate transfer reactions that utilize adenosine triphosphate (ATP) and other nucleotide triphosphates as substrates. Various phosphatases and pyrophosphatases also represent enzymes from a list that is influenced by this metallic ion.

Magnesium plays a role in the reversible association of intracellular particles and in the binding of macromolecules to subcellular organelles. For example, the binding of messenger RNA (mRNA) to ribosomes is magnesium dependent, as is the functional integrity of ribosomal subunits. Certain effects of magnesium on the nervous system are similar to those of calcium. An increased concentration of magnesium in the extracellular fluid can cause depression of the central nervous system (CNS). Hypomagnesemia can cause increased CNS irritability, disorientation, and convulsions. Magnesium also has a direct depressant effect on skeletal muscle. Abnormally low concentrations of magnesium in the extracellular fluid can result in increased acetylcholine release and increased muscle excitability that can produce tetany. Magnesium can also aid in managing symptoms of PMS and aids in the methylation support of estrogenic metabolites.

Magnesium can be present in the preferred embodiments as inorganic salts, organic salts, or amino acid chelates, or the like, or mixtures thereof. Preferred forms of magnesium include magnesium glycinate, magnesium citrate, and magnesium ascorbate.

Trace Elements

Chromium is a trace element wherein the lack of sufficient chromium in the diet leads to impairment of glucose utilization; however, disturbances in protein and lipid metabolism have also been observed with lack of sufficient chromium. Impaired glucose utilization occurs in many middle-aged and elderly human beings. In experimental studies, significant numbers of such persons have shown improvement in their glucose utilization after treatment with chromium. Chromium is transported by transferring in the plasma and competes with iron for binding sites. Chromium as a dietary supplement can produce benefits due to its enhancement of glucose utilization and its possible facilitating the binding of insulin to insulin receptors, which increases its effects on carbohydrate and lipid metabolism. Chromium as a supplement can produce benefits in conditions, such as, but not limited to, atherosclerosis, diabetes, rheumatism, and weight control. A preferred form of chromium according to the preferred embodiments comprises chromium polynicotinate.

Copper is another trace element in the diet. A common defect observed in copper-deficient animals is anemia. Other abnormalities due to copper deficiency include, but not limited to, growth depression, skeletal defects, demyelination and degeneration of the nervous system, ataxia, defects in pigmentation and structure of hair or wool, reproductive failure and cardiovascular lesions, including dissecting aneurysms. Several copper-containing metalloproteins have been isolated, including tyrosinase, ascorbic acid oxidase, lactase, cytochrome oxidase, uricase, monoamine oxidase, δ-aminolevulinic acid hydrydase, and dopamine-β-hydroxylase. Copper functions in the absorption and utilization of iron, electron transport, connective tissue metabolism, phospholipid formation, purine metabolism, and development of the nervous system. Ferroxidase I (ceruloplasmin), a copper-containing enzyme, effects the oxidation of Fe (II) to Fe (III), a step for mobilization of stored iron. A copper-containing enzyme is thought to be responsible for the oxidative deamination of the epsilon amino group of lysine to produce desmosine and isodesmosine, the cross-links of elastin. In copper-deficient animals, the arterial elastin is weaker and dissecting aneurysms can occur. A preferred form of copper according to the preferred embodiments comprises copper gluconate.

Iodine is used for the production of thyroid hormones, which regulate cellular oxidation. An iodine-deficiency disease is goiter. In iodine-deficient young, growth is depressed and sexual development is delayed, the skin and hair are typically rough, and the hair becomes thin. Cretinism, feeble-mindedness, and deaf-mutism occur in a severe deficiency. There is reproductive failure in females and decreased fertility in males that lack sufficient iodine in the diet. A preferred form of iodine according to the preferred embodiments comprises potassium iodide.

Molybdenum is a mineral found in high concentrations in the liver, kidneys, skin, and bones. This mineral is used by the body to properly metabolize nitrogen. It is also a component of the enzyme xanthine oxidase, which is used to convert purines to uric acid, a normal byproduct of metabolism. Molybdenum also supports the body's storage of iron and other cellular functions, such as growth. A deficiency of molybdenum is associated with mouth and gum disorders and cancer. A diet high in refined and processed foods can lead to a deficiency of molybdenum, resulting in conditions such as, but not limited to, anemia, loss of appetite and weight, and stunted growth in animals. While these deficiencies have not been observed directly in humans, it is known that a molybdenum deficiency can lead to impotence in older males. A preferred form of molybdenum according to the preferred embodiments comprises molybdenum amino acid chelate.

Selenium is a trace element that functions as a component of enzymes involved in protection against antioxidants and thyroid hormone metabolism. In several intra-and extra-cellular glutathione peroxidases and iodothyronine 5′-deiodinases, selenium is located at the active centers as the selenoamino acid, selenocysteine (SeCys). At least two other proteins of unknown function also contain SeCys. Although SeCys is an important dietary form, it is not directly incorporated into these specific selenium-proteins; instead, a co-translational process yields tRNA-bound SeCys. In contrast, selenium as seleno-methionine is incorporated non-specifically into many proteins, as it competes with methionine in general protein synthesis. Therefore, tissues often contain both specific, as well as the nonspecific, selenium-containing proteins when both SeCys and selenomethionine are consumed, as found in many foods. Selenium is a major antioxidant nutrient and is involved in protecting cell membranes and preventing free radical generation, thereby decreasing the risk of cancer and disease of the heart and blood vessels. Medical surveys show that increased selenium intake decreases the risk of breast, colon, lung and prostate cancers. Selenium can also preserve tissue elasticity; slow down the aging and hardening of tissues through oxidation; and help in the treatment and prevention of dandruff. Recent research has shown antitumorigenic effects of high levels of selenium in the diets of several animal models. A preferred form of selenium according to the preferred embodiments comprises selenium amino acid complex.

Zinc is known to occur in many important metalloenzymes. These metalloenzymes include, but are not limited to, carbonic anhydrase, carboxypeptidases A and B, alcohol dehydrogenase, glutamic dehydrogenase, D-glyceraldehyde-3-phosphate dehydrogenase, lactic dehydrogenase, malic dehydrogenase, alkaline phosphatase, and aldolase. Impaired synthesis of nucleic acids and proteins has been observed in zinc deficiency. There is also evidence that zinc can be involved in the secretion of insulin and in the function of the hormone. A preferred form of zinc according to the preferred embodiments comprises zinc citrate.

Magnesium, calcium, and vitamin E and supplementation with these ingredients are associated with significant improvement in premenstrual symptoms. (R. A. Sherwood et al., Magnesium and the Premenstrual Syndrome, 23 Ann. Clin. Biochem. 667-670 (1986); A. Bendich, The Potential for Dietary Supplements to Reduce Premenstrual Syndrome (PMS) Symptoms, 19 J. Am. Coll. Nutr. 3-12 (2000); R. S. London et al., Efficacy of Alpha-tocopherol in the Treatment of the Premenstrual Syndrome, 32 J. Reprod. Med. 400-404 (1987))

Preferred formulations and ranges of these ingredients in the preferred embodiments are shown in Table 4 below.

TABLE 4
Preferred Formulations and Ranges of Minerals

Ranges in Parts by Weight

| Minerals | Preferred | More Preferred |
|---|---|---|
| Calcium | 10-2,000 × 10⁻³ | 100-1,500 × 10⁻³ |
| Magnesium | 50-1,000 × 10⁻³ | 100-800 × 10⁻³ |
| Chromium | 10-500 × 10⁻⁶ | 0-300 × 10⁻⁶ |
| Copper | 0.1-10 × 10⁻³ | 0.5-5 × 10⁻³ |
| Iodine | 10-500 × 10⁻⁶ | 10-300 × 10⁻⁶ |
| Iron | 0.1-100 × 10⁻³ | 1-50 × 10⁻³ |
| Phosphorus | 10-1000 × 10⁻³ | 100-750 × 10⁻³ |
| Molybdenum | 5-500 × 10⁻⁶ | 10-200 × 10⁻³ |
| Selenium | 2-1,000 × 10⁻⁶ | 10-500 × 10⁻⁶ |
| Zinc | 0.1-200 × 10⁻³ | 1-100 × 10⁻³ |
| Manganese | 0.1-25 × 10⁻³ | 0.5-10 × 10⁻³ |
| Sodium | 0.1-200 × 10⁻³ | 1-100 × 10⁻³ |
| Potassium | 10-1000 × 10⁻³ | 100-600 × 10⁻³ |

According to the preferred embodiments, minerals can be provided as inorganic compounds, such as chlorides, sulfates, and the like. In addition, some minerals can be provided in more bioavailable forms, such as amino acid chelates, which are well known in the art, as disclosed in U.S. Pat. No. 5,292,538 and incorporated herein by reference. Examples of minerals that can be provided as amino acid chelates include, but are not limited to, calcium, magnesium, manganese, zinc, iron, boron, copper, molybdenum, and chromium.

In addition to the above-identified minerals, it is also beneficial to include such minerals as potassium phosphate and tetrasodium phosphate for their usual salutary effects.

Amino Acids

Amino acids, or more precisely, α-amino acids, are the fundamental structural units of proteins. Twenty amino acids are commonly found in proteins. The nutritional value of proteins in our diet involves recognition of the quality, as well as the quantity, of the protein. Humans do not have the ability to synthesize all the amino acids required for normal good health. Amino acids that are supplied by the diet are called essential amino acids and include leucine, isoleucine, lysine, methionine, phenylalanine, threonine, tryptophan, and valine. In general, it is recommended that an adult should take in about 10 grams or protein per kilogram of body weight each day. Children require about 2-3 times this amount. Of course, this recommendation assumes that the protein in the diet has an adequate amount of all essential and nonessential amino acids.

To ensure that all of the essential amino acids are present in the diet in adequate amounts, the medical composition of the preferred embodiments includes, but is not limited to, the following amino acids: lysine, cysteine, and threonine. In addition, the modified amino acid, N-acetylcysteine is used for the synthesis of glutathione, thus supporting the glutathione conjugation detoxification pathway. (C. H. Yim et al., Use of N-acetylcysteine to Increase Intracellular Glutathione During the Induction of Antitumor Responses by IL-2, 152 J. Immunol. 5796-5805 (1994); D. J. Liska et al., Detoxification: A Clinical Monograph (Institute for Functional Medicine. Gig Harbor, Wash. 1999)) Additionally, N-acetylcysteine supports phase II sulfation, an important step in estrogen detoxification. (G. Levy, Sulfate Conjugation in Drug Metabolism: Role of Inorganic Sulfate, 45 Federation Proc. 2235-2240 (1986)) Sulfation can be a route of elimination of estrogenic compounds. Accordingly, it is preferably to include N-acetylcysteine in the preferred embodiments to aid in sulfation of estrogenic compounds.

The modified amino acid, trimethylglycine (betaine), is also advantageously added to the medical composition of the preferred embodiments, preferably in an amount of about 1-500×10⁻³ parts by weight, and more preferably about 100-300×10⁻³ parts by weight. Choline, betaine, and pyridoxine are included for their ability to provide methylation support. Methylation of the catechol estrogens (2-OH and 4-OH) via the catechol-O-methyltransferase enzyme is the principal means of deactivation. This reaction requires S-adenosylmethionine (SAM), which is converted to homocysteine (HCys). Nutrients to support the methylation cycle may support detoxification of the catechol estrogens as well as help maintain healthy HCys levels. (M. Butterworth et al., 17-β-Estradiol Metabolism by Hamster Hepatic Microsomes, implications for the Catechol-O-Methyl Transferase-mediated Detoxification of Catechol Estrogens, 24 Drug Metab. Dispos. 588-594 (1996); C. E. Garner et al., Catechol Metabolites of Polychlorinated Biphenyls Inhibit the Catechol-O-Methyltransferase-mediated Metabolism of Catechol Estrogens, 162 Toxicol. Appl. Pharmacol. 115-123 (2000)) Some data suggest that post-menopausal women routinely have elevated serum HCys levels. (K. Zhu & S. M. Williams, Methyl-deficient Diets, Methylated ER Genes and Breast Cancer: An Hypothesized Association, 9 Cancer Causes Control 615-620 (1998); A. Andersson et al., Plasma Homocysteine Before and After Methionine Loading with Regard to Age, Gender, and Menopausal Status, 22 Eur. J. Clin. Invest. 79-87 (1992))

Preferred formulations and ranges of these fortifying ingredients in the preferred embodiments are shown in Table 5 below.

TABLE 5
Preferred Formulations and Ranges of Amino Acids

Ranges in Parts by Weight

| Amino Acids | Preferred | More Preferred |
|---|---|---|
| L-Lysine | 0.1-100 × 10⁻³ | 1-50 × 10⁻³ |
| L-threonine | 0.1-100 × 10⁻³ | 1-50 × 10⁻³ |
| trimethylglycine | 0.1-1000 × 10⁻³ | 1-500− × 10⁻³ |
| N-acetylcysteine | 0.1-500 × 10⁻³ | 1-200 × 10⁻³ |

Carotenoids

Carotenoids are a family of hundreds of plant pigments found in fruits and vegetables that are red, orange, and deep yellow in color, and also in some dark green leafy vegetables. See USDA-NCC Carotenoid Database for U.S. Foods (1998). Carotenoids are the precursors of most of the vitamin A found in animals. At least about 10 different carotenoids exhibit provitamin A activity, including α and β-carotenes and cryptoxanthin. As precursors of vitamin A, carotenoids can exhibit an effect on vision, but carotenoids are known to have other beneficial effects in the diet, as well. For example, carotenoids are also known for their antioxidant activity in helping protect the body from free radical damage. Certain embodiments comprise about 10-8000 IU, and more preferably about 150-4000 IU of β-carotene as mixed carotenoids.

Volumes of research reveal that two carotenoids—lutein and zeaxanthin—are found in significant concentrations in the macula of the eye. This research also indicates that maintaining significant levels of these two carotenoids, particularly lutein, can help diminish the effects of age-related macular degeneration, the leading cause of blindness in those over about 65 years of age. Lutein can act as an antioxidant and protect cells against the damaging effects of free radicals. As with the other carotenoids, lutein is not made in the body and, therefore, can be obtained from food or dietary supplements.

At one time, researchers believed all antioxidants served the substantially the same purpose. Now, there is growing evidence that individual antioxidants can be used by the body for specific purposes. Researchers believe that lutein is deposited into areas of the body most prone to free radical damage. One major example is the macula, a tiny portion of the retina. Research indicates that because of its antioxidant properties, lutein consumption can play a role in maintaining the health of the eyes, heart and skin as well as the breasts and cervix in women. In addition, scientists are studying lutein's possible role in conditions such as, but not limited to, age-related macular degeneration, cataracts, heart disease, and immune system health. Studies have also shown that lutein is associated with a reduction in lung, breast, and cervical cancer. In the vascular system, lutein is found in high-density lipoprotein (“HDL”) or “good” cholesterol and can prevent low-density lipoprotein (“LDL”) or “bad” cholesterol from oxidizing, which sets a cascade for heart disease.

Besides being a precursor of vitamin A, β-carotene is thought to be effective in helping to protect against some diseases, such as, but not limited to, cancer, heart disease, and stroke.

Lycopene is an open-chain unsaturated carotenoid that imparts red color to foods such as, but not limited to, tomatoes, guava, rosehip, watermelon, and pink grapefruit. Lycopene is a proven anti-oxidant that can lower the risk of certain diseases including cancer and heart disease. In the body, lycopene is deposited in the liver, lungs, prostate gland, colon, and skin. Its concentration in body tissues tends to be higher than all other carotenoids. Epidemiological studies have shown that high intake of lycopene-containing vegetables is inversely associated with the incidence of certain types of cancer. For example, habitual intake of tomato products has been found to decrease the risk of cancer of the digestive tract, as seen among Italians who ingest high amount of tomato products. In a six-year study by Harvard Medical School and Harvard School of Public Health, the diets of more than about 47,000 men were studied. Of forty-six fruits and vegetables evaluated, tomato products (which contain large quantities of lycopene) showed a measurable relationship to reduce prostate cancer risk. As consumption of tomato products increased, levels of lycopene in the blood increased, and the risk for prostate cancer decreased. Ongoing research suggests that lycopene can reduce the risk of macular degenerative disease, serum lipid oxidation, and cancers of the lung, bladder, cervix and skin. Studies are underway to investigate other potential benefits of lycopene, including lycopene's potential in the fight against cancers of the digestive tract, breast, and prostate. (W. Stahl & H. Sies, Lycopene: a biologically important carotenoid for humans? 336 Arch. Biochem. Biophys. 1-9 (1996); H. Gerster, The potential role of lycopene for human health, 16 J. Amer. Coll. Nutr. 109-126 (1997))

Other Beneficial Phytonutrients

There are many other naturally occurring compounds derived from a variety of plant sources that promote healthy estrogen metabolism. Many antioxidant nutrients and phytonutrients can reduce the oxidation of catechol estrogen metabolites into quinones. Notable players in this group include vitamins E and C, α-lipoic acid, N-acetylcysteine, the mineral selenium, curcumin, and green tea polyphenols.

D-limonene, a naturally occurring monoterpene found in the oils of citrus fruits, promotes the detoxification of estrogen by inducing Phase I and Phase II enzymes in the liver, including GST. This compound has also shown great promise in the prevention and treatment of breast and other cancers.

There are also many hormone-modulating herbs that have a long history of traditional use in treating women's health conditions. These include black cohosh (Cimicifuga racemosa), chasteberry (Vitex agnus castus), ginseng (Panax ginseng), dong quai (Angelica sinensis), and licorice (Glycyrrhiza uralensis). While the mechanism of action of these herbs in promoting healthy estrogen balance varies, many have been found to contain phytoestrogens.

Other Ingredients

Preferably, the present medical composition of the preferred embodiments further comprises natural flavors, formulation aids (such as xanthan, carrageenan, and cellulose gum), and the like for their usual beneficial properties.

The preferred embodiments advantageously further comprises glutathione and ferrochel amino acid chelate.

Other Effects

In a pathway, SAM can function as a methyl group donor for a range of compounds. As a result of intervening at a pathway, a bodily process can be affected. Then, as a result of affecting a bodily process, conditions or diseases corresponding to the bodily process can be treated or prevented. For example, intervention of the pathway of methylation of estrogen can result in managing the bodily process of balancing hormones. As a result of balancing hormones, disease or conditions, such as premenstrual syndrome, can be treated or prevented.

As mentioned above, SAM is a co-factor to COMT for sterol methylation. In addition to COMT, another enzyme that can affect sterol methylation is S-adenosyl-L-methionine:delta-24[25]sterol methyltransferase. However, other enzymes that utilize SAM are contemplated and the use of the components of medical composition for affecting other bodily processes through utilization of SAM is considered to be within the scope.

The medical composition affects the levels of S-adenosylmethionine (SAM), which is a cofactor that can transfer one-carbon groups with the help of enzymes. SAM is naturally synthesized in the body during the metabolism of methionine to cysteine, taurine, glutathione, and other polyamine compounds. SAM exists in varying amounts in mammalian cells. Although synthesized in many cells, the majority of SAM's generation is in the liver. As a cofactor for use in a pathway, SAM functions as a methyl group donor for a range of compounds. For example, the use of the components of the medical composition can affect the methylation of compounds including, but not limited to, catecholamines, neurotransmitters, proteins, membrane phospholipids, fatty acids, nucleic acids, porphyrins, choline, camitine, creatine, and hormones, including peptide hormones, amine hormones, steroid hormones, eicosanoids, and the like.

Since the medical composition can affect levels of SAM, there is wide potential of affecting a variety of bodily processes that utilize SAM in the pathway. Described above is SAM affecting COMT or S-adenosyl-L-methionine:delta-24[25]sterol methyltransferase to ultimately affect hormone balance. As a result of balancing hormones, conditions or diseases, such as, but not limited to, cancer, premenstrual syndrome, endometriosis, uterine fibroid tumors, fibrocystic or painful breasts, cervical dysplasia, systemic lupus erythematosis, vaginitis, fatigue, cognitive dysfunction, depression, and irritability, can be treated or prevented.

One methylation process involving SAM as a co-factor is DNA methylation. DNA methylation, or the covalent addition of methyl groups to cytosine, has profound effects on the genome. These effects include, but are not limited to, transcriptional repression by inhibition of transcriptional factor binding, or recruitment of methyl binding proteins and their associated chromatin remodeling factors. DNA methylation is also used for embryonic development. Normal methylation patterns are frequently disrupted in tumor cells with global hypomethylation accompanying region-specific hypermethylation. Hence, DNA methylation can have significant clinical impact on the reduction of risk for a number of age-related and other diseases, including, but not limited to, cancer, liver damage, and brain cell degeneration.

Other conditions or diseases that can be treated by affecting by levels of SAM include, but are not limited to, various depressive disorders, such as depression; osteoarthritis; fibromyalgia; gastrointestinal injury, liver dysfunction. Increased levels of SAM has been shown to give beneficial effects to conditions, such as migraine, Parkinson's disease, Alzheimer's disease, organic brain syndrome, epilepsy, HIV-related neurologic complications, multiple sclerosis, metabolic defects, and spinal cord disease.

Formulations

The medical composition of the preferred embodiments is preferably formulated as a powder. The ingredients can be combined and mixed into a homogeneous powdered mixture. This powdered mixture is then packaged in any convenient packing material known in the art. The powdered mixture can be added to water or juice; mixed; and then taken orally as a meal replacement. The medical food can also be formulated into a dietary bar, dietary gel, and the like.

Alternatively, the medical composition can be administered by mouth in the form of tablets, capsules, solutions, emulsions, or suspensions. The medical composition can additionally contain preservatives, solubilizers, stabilizers, wetting agents, emulsifiers, sweeteners, colorants, flavorings, buffers, coating agents, and antioxidants.

The disclosure below is of specific examples setting forth preferred embodiments. These examples are not intended to limit the scope, but rather to exemplify preferred embodiments.

### EXAMPLE 1

Preparation of Medical Composition in the Form of Medical Food

A medical food was designed for nutritional support of women with symptoms associated with hormone cycles. The nutrient profile of the medical food is shown in Table 6. The amounts shown in Table 6 can be decreased by two-fold or increased by two-fold. Specifically, the medical food was designed with specific rice macronutrients of low-allergy potential to provide protein and carbohydrates, and flax meal to provide lignin, a fiber that shows specificity for binding steroid hormones, and lignan, a source of phytoestrogens.

TABLE 6
Composition of the medical food for nutritional support
of symptoms related to hormone cycles, provided as
nutrients delivered in two servings per day.

| | Amount per day | |
|---|---|---|
| Macronutrients | | |
| Protein | 30 | g |
| Fiber | 8 | g |
| Carbohydrates | 46 | g |
| Fat | 6 | g |
| Micronutrients | | |
| Vitamin A/Mixed Components (carotenoids) | 7500 | IU |
| Vitamin C | 120 | mg |
| Vitamin D | 400 | IU |
| Vitamin E | 600 | IU |
| Vitamin K | 80 | mcg |
| Thiamin (B1) | 1.5 | mg |
| Riboflavin (B2) | 1.7 | mg |
| Niacin (B3) | 20 | mg |
| Vitamin B6 | 100 | mg |
| Vitamin B12 | 60 | mcg |
| Biotin | 300 | mcg |
| Folic Acid | 1 | mg |
| Pantothenic Acid | 10 | mg |
| Phosphorus | 520 | mg |
| Iron | 18 | mg |
| Calcium | 700 | mg |
| Iodine | 150 | mcg |
| Magnesium | 480 | mg |
| Zinc | 15 | mg |
| Selenium | 70 | mcg |
| Copper | 2 | mg |
| Manganese | 2 | mg |
| Chromium | 200 | mcg |
| Molybdenum | 75 | mcg |
| Isoflavones (from kudzu) | 50 | mg |
| Choline | 500 | mg |
| Curcumin | 400 | mg |
| Trimethylglycine | 400 | mg |
| N-Acetylcysteine | 200 | mg |

The medical food was fortified with a nutrient core that included a non-soy source of isoflavones, which modifies effects of endogenous estrogen; the phytonutrient curcumin, which has long been shown to act synergistically with the isoflavone genistein; enhanced levels of B vitamins with choline, trimethylglycine and N-acetylcysteine, which support estrogen detoxification and methylation metabolic pathways; and magnesium, calcium, and vitamin E, which have been associated with amelioration of PMS symptoms.

### EXAMPLE 2

Preparation of Medical Composition in the Form of Tablet

A medical composition in the form of tablets was designed for nutritional support of women with symptoms associated with hormone cycles. The nutrient profile of the medical composition is shown in Table 7. The amounts shown in Table 6 can be decreased by two-fold or increased by two-fold.

TABLE 7
Composition of the medical composition in tablet form for
nutritional support of symptoms related to hormone cycles,
provided as nutrients delivered in two servings per day.

| Micronutrients | Amount per day | |
|---|---|---|
| Vitamin A/Mixed Components | 2500 | IU |
| Vitamin D | 200 | IU |
| Vitamin E | 200 | IU |
| Vitamin K | 40 | mcg |
| Vitamin B6 | 50 | mg |
| Vitamin B12 | 30 | mcg |
| Folic Acid | 800 | mcg |
| Isoflavones (from kudzu) | 100 | mg |
| Curcumin | 200 | mg |
| Trimethylglycine | 200 | mg |
| Resveretrol | 2 | mg |
| Rosemary extract | 200 | mg |
| Chrysin | 100 | mg |

### EXAMPLE 3

Clinical Study of the Effects of Medical Food on PMS Symptoms

The clinical trial was performed at the Functional Medicine Research Center, Gig Harbor, Wash. The inclusion criteria for the study were women between 21 and 45 years of age who were exhibiting significant symptoms of PMS as assessed by scores on a PMS symptoms-specific questionnaire (Shortened Premenstrual Assessment Form, described below). Exclusion criteria for the study included: evidence of untreated endocrine, neurological, or infectious disorder; pregnancy or lactation; history of diabetes, mental illness or attempted suicide, or liver, kidney or heart disease; use of oral corticosteroids within four weeks prior to the screening; use of anti-arrhythmic or other cardiac medications.

The study was a boxed, 2-armed trial with stratification based on the use of birth control medication. Primary endpoints were monitored at the Screening Visit, Visit 1, Visit 2, and Visit 3. At Visit 1, subjects were randomized and baseline serum and urine were obtained. All visits were planned at the time when each woman was in the luteal phase of her cycle (i.e., at 75-80% through the subject's usual menstrual cycle). The trial lasted for three complete menstrual cycles.

All subjects were randomly assigned to Group A [medical food and a capsule containing the phytonutrient indole-3-carbinol ( I3C)], or Group B [medical food and a placebo capsule]. The medical food used in the trial is presented in Example 1 of this application. Both clinicians and subjects were blinded regarding the randomization. (The I3C was included in the study to determine if additional benefit could be achieved from targeted nutritional modulation of detoxification activities.)

Clinical Assessment

Two research-validated, PMS-specific questionnaires were chosen for monitoring PMS symptoms: the Shortened Premenstrual Assessment Form (SPAF) and the Menstrual Distress Questionnaire (MDQ). The SPAF rates symptoms in the second half of a woman's menstrual cycle on a scale of 1 through 6 (1=no symptoms; 6=extreme symptoms). The MDQ uses a five-point scale (0=no symptoms; 4=severe symptoms), and rates symptoms for three different stages of the menstrual cycle; premenstrual (4 days before most recent flow); menstrual (most recent flow); and intermenstrual (remainder of cycle). The MDQ data is transformed to provide a normalized score for which a population mean of 50 and a standard deviation of 10 have been determined as reference values. Scores higher than 50±10 indicate PMS symptoms are present.

Subjects were also asked to fill out the Medical Outcomes Survey SF-36 questionnaire, a well-validated, quality-of-life instrument. Information on symptoms and medication use, as well as assessment of compliance to the protocol, was obtained at each visit.

Laboratory Assessment

Aspartate aminotransferase, alanine aminotransferase, bilirubin, urea nitrogen, creatinine, albumin, and glucose were assessed by standard photometric methods from 10-12 hour fasting serum obtained at the Screening Visit and Visit 3. The following were preformed on 10-12 hour fasting serum from Visit 1 and Visit 3 (Laboratories Northwest, Tacoma, Wash.): photometric measurements of triglycerides, and total-, HDL- and LDL-cholesterol; radioimmunoassay measurements of SHBG, progesterone and testosterone; automated chemiluminescence analysis (DPC Immulite 2000) of bound estradiol; and high performance liquid chromatography quantification of homocysteine. Urinary estrogen metabolites (estradiol, estrone, and estriol) were obtained from a 24-hour urine collection at Visit 1 and Visit 3, and were quantified by gas chromatography/mass spectrophotometric methods (AAL Reference Laboratories, Santa Ana, Calif.). Total estrogen excretion was determined by addition of the 24-hour excretion of the 3 estrogen metabolites.

Statistical Analysis

Baseline data (the level of symptoms experienced in the preceding 2 menstrual cycles) were obtained from averaging the Screening Visit and Visit 1 values, and served as a control for non-intervention variability. Laboratory and questionnaire data were analyzed by a one-way analysis of variance (ANOVA) using JMP Statistical Package (SAS Institute, Cary, N.C.). Variances in laboratory analyses were determined using split sample analysis.

Results

Fifty-one subjects qualified for the trial; eight of these dropped out of the trial after the initial screening but prior to any intervention. Therefore, forty-three subjects began the clinical trial; of these, three subjects were withdrawn from the trial during the course of the intervention (one subject withdrew for unknown reasons, but commented on the taste of the medical food, and two subjects experienced adverse symptoms that included gastrointestinal pain and diarrhea). Forty subjects, between the ages of 21-45 (average 36±6 years), completed the clinical trial.

Subjects showed laboratory values within the normative reference range for liver and kidney function prior to, and after the intervention with the medical food (Table 8). Alanine aminotransferase appeared to increase after intervention; however, more variability was observed in the post intervention value, and both pre- and post-intervention were well within the reference range. Lipid panel and blood glucose assessments from 10-12 hour fasting serum were also within normative values and displayed no change following intervention.

TABLE 8
General laboratory markers for subjects

| | Reference | Mean (± sem) | | |
|---|---|---|---|---|
| | Range | Baseline | Final | p |
| Total cholesterol | 120-200 | 182 (4.9) | 190 (5.0) | ns |
| (mg/dL)* | | | | |
| HDL (mg/dL)* | 55-70 | 51 (2.1 | 55 (2.2) | ns |
| LDL (mg/dL)* | 80-130 | 111 (4.4) | 115 (4.5) | ns |
| Triglycerides | 10-175 | 104 (8.2) | 104 (8.1) | ns |
| (mg/dL)* | | | | |
| Glucose | 65-120 | 85 (1.9) | 87 (1.4) | ns |
| (mg/dL)# | | | | |
| Albumin (g/dL)* | 3.2-5.0 | 3.8 (0.04) | 3.8 (0.03) | ns |
| Bilirubin | 0.0-1.4 | 0.26 (0.03) | 0.34 (0.03) | ns |
| (mg/dL)# | | | | |
| Urea nitrogen | 8-24 | 13 (0.6) | 12 (0.5) | ns |
| (mg/dL)# | | | | |
| Creatinine | 0.6-1.2 | 0.76 (0.10) | 0.78 (0.10) | ns |
| (mg/dL)# | | | | |
| Aspartate | 10-56 | 22 (0.7) | 24 (1.2) | 0.08 |
| aminotransferase | | | | |
| (IU/L)# | | | | |
| Alanine | 5-60 | 22 (0.8) | 30 (1.7) | <0.01 |
| aminotransferase | | | | |
| (IU/L)# | | | | |

*N = 39;
#N = 40;
p = significance

The questionnaire data showed no difference between the medical food/13C group and the medical food/placebo group, therefore, questionnaire results for the 2 treatment groups were pooled for the purpose of analysis. Eleven patients were on oral birth control pills; however, since no differences were noted between those on birth control and those not on birth control, these data were pooled as well.

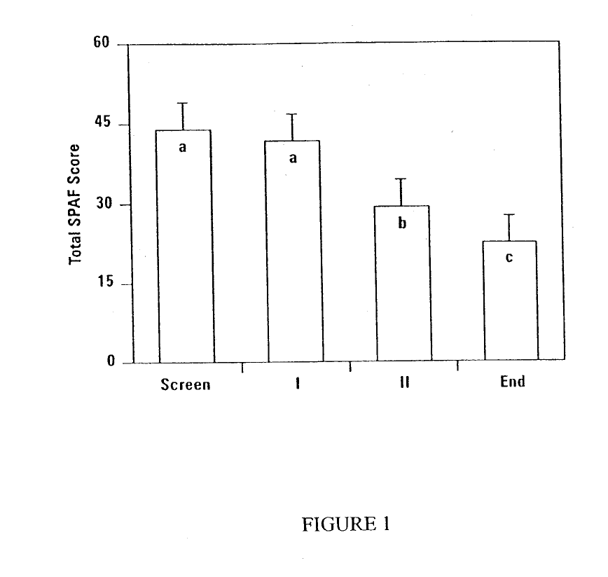

The SPAF provides a score for total overall symptoms, as well as 3 subscores for pain, water retention, and negative affect. Subjects showed no significant change in symptoms during the 2 cycles of the base line time course; the Screening Visit and Visit 1 average scores were 44.6 and 41.7, respectively. After beginning the intervention with the medical food, the subjects reported an average total score for symptoms of 29.3 at Visit 2, and 22.9 at Visit 3, which is about 59% reduction in symptoms with a statistical significance of p<0.05. These results are graphically depicted in FIG. 1. Significant decreases were consistently observed in all categories of the SPAF (Table 9), with improvements of the subscores for pain, water retention, and negative affect of about 61%, 58%, and 61%, respectively (p<0.05).

TABLE 9
Mean changes (± sd) in Shortened Premenstrual Assessment
Form (SPAF) scores after intervention with medical food
in subjects with PMS symptoms (n = 38)

| SPAF | | | | |
|---|---|---|---|---|
| Category | Screening Visit | Visit 1 | Visit 2 | Visit 3 |
| Affect | 20.6 (2.6)^{a} | 18.3 (3.2)^{a} | 13.3 (4.9)^{b} | 9.6 (4.7)^{c} |
| Pain | 12.3 (3.5)^{a} | 11.5 (3.0)^{a} | 8.0 (3.2)^{b} | 6.6 (2.4)^{b} |
| Water | 12.6 (3.6)^{a} | 11.8 (3.7)^{a} | 8.6 (3.4)^{b} | 6.6 (2.8)^{b} |
| Retention | | | | |
| SPAF Total | 44.6 (9.4)^{a} | 41.7 (8.0)^{a} | 29.3 (10.4)^{b} | 22.9 (8.3)^{c} |
| Score | | | | |

The total SPAF score is pooled data from the three subsections. Incomplete questionnaires were not included in the analysis.
Entries share a superscript (a, b, or c) if they do not differ significantly (a = 0.05) from each other. Sequential letters indicate a difference of p < 0.05 from the preceding value.

The MDQ provides a more detailed assessment of PMS symptoms, which are presented in 7 PMS symptom-specific subcategories (pain, water retention, autonomic reactions, negative affect, impaired concentration, behavior change, and arousal) and 1 control subcategory for 3 different times during a woman's cycle (internenstrual, menstrual, and premenstrual). The control category contains questions that have been reported more frequently by menopausal women but are infrequently reported by premenopausal woman and has been included in the questionnaire as an internal control for a woman's tendency to report symptoms that may not be related to PMS. Table 10 shows the mean (±sem) for the subjects' responses to the different symptom categories of the MDQ during the intervention.

TABLE 10
Mean (+ sem) of Menstrual Distress Questionnaire (MDQ) results
of PMS symptoms for forty subjects on the medical food

| | | | | | |
|---|---|---|---|---|---|
| | | | | | Signif- |
| Screening | | | | | icance |
| Category | Visit | Visit 1 | Visit 2 | Visit 3 | (p) |

Pain

| | | | | | |
|---|---|---|---|---|---|
| Intermenstrual | 69.8 (5.2) | 62.0 (3.8) | 58.3 (2.8) | 56.8 (3.0) | 0.0753 |
| Menstrual | 73.0 (3.2) | 72.0 (3.4) | 55.4 (2.5) | 53.5 (2.6) | <0.0001 |
| Premenstrual | 81.4 (3.2) | 76.8 (3.5) | 63.1 (2.6) | 57.1 (2.9) | <0.0001 |

Water Retention

| | | | | | |
|---|---|---|---|---|---|
| Intermenstrual | 69.1 (6.0) | 61.1 (4.6) | 55.6 (3.3) | 53.8 (2.7) | 0.0595 |
| Menstrual | 74.6 (3.3) | 71.6 (3.4) | 56.8 (2.6) | 51.8 (2.3) | <0.0001 |
| Premenstrual | 83.4 (2.9) | 81.2 (3.7) | 64.5 (3.1) | 58.2 (2.6) | <0.0001 |

Autonomic Reactions

| | | | | | |
|---|---|---|---|---|---|
| Intermenstrual | 56.4 (6.9) | 45.4 (3.9) | 45.1 (3.8) | 41.4 (2.8) | 0.1212 |
| Menstrual | 69.1 (4.7) | 64.1 (4.7) | 53.4 (3.1) | 50.3 (2.2) | 0.0014 |
| Premenstrual | 75.2 (5.2) | 68.7 (4.8) | 57.5 (3.5) | 53.3 (2.5) | 0.0007 |

Negative Affect

| | | | | | |
|---|---|---|---|---|---|
| Intermenstrual | 73.5 (5.6) | 64.2 (4.3) | 54.5 (2.9) | 56.0 (3.1) | 0.0045 |
| Menstrual | 78.3 (3.8) | 76.7 (3.6) | 58.4 (3.1) | 52.8 (2.6) | <0.0001 |
| Premenstrual | 90.5 (2.2) | 84.7 (2.6) | 63.2 (2.6) | 55.3 (2.4) | <0.0001 |

Impaired Concentration

| | | | | | |
|---|---|---|---|---|---|
| Intermenstrual | 68.3 (4.6) | 61.0 (2.8) | 56.8 (2.6) | 54.5 (2.6) | 0.0187 |
| Menstrual | 78.0 (5.6) | 79.7 (5.5) | 60.0 (3.8) | 56.1 (3.1) | 0.0002 |
| Premenstrual | 88.0 (5.5) | 87.5 (4.7) | 65.8 (3.5) | 61.4 (3.6) | <0.0001 |

Behavior Change

| | | | | | |
|---|---|---|---|---|---|
| Intermenstrual | 67.0 (5.4) | 59.3 (3.7) | 53.4 (2.4) | 54.4 (3.0) | 0.0461 |
| Menstrual | 71.4 (4.1) | 69.3 (4.1) | 53.3 (2.3) | 48.7 (2.2) | <0.0001 |
| Premenstrual | 86.4 (5.6) | 77.5 (4.4) | 59.5 (2.9) | 54.5 (3.2) | <0.0001 |

Arousal

| | | | | | |
|---|---|---|---|---|---|
| Intermenstrual | 60.5 (3.1) | 57.3 (2.7) | 56.7 (2.6) | 51.4 (2.3) | 0.1242 |
| Menstrual | 55.7 (2.2) | 54.2 (2.3) | 55.7 (2.4) | 49.7 (2.3) | 0.2091 |
| Premenstrual | 53.9 (3.1) | 56.2 (2.6) | 55.4 (2.4) | 50.1 (2.2) | 0.3519 |

Control

| Intermenstrual | 63.6 (4.6) | 58.7 (4.2) | 58.4 (5.5) | 53.5 (3.6) | 0.4723 |
|---|---|---|---|---|---|
| Menstrual | 62.6 (3.3) | 63.7 (5.2) | 53.1 (3.3) | 51.1 (2.2) | 0.0286 |
| Premenstrual | 71.1 (4.6) | 70.0 (4.2) | 58.3 (4.2) | 53.8 (2.9) | 0.0111 |
| The data are presented for the seven categories of PMS symptoms and the control category, which rates symptoms not generally | | | | | |
| # associated with PMS as an internal control for intermenstrual, menstrual, and premenstrual times during each cycle. The scores are | | | | | |
| # presented as T-scores, which for the population have a mean of 50 and a standard deviation of 10. The significance (p) was obtained | | | | | |
| # from ANOVA analysis. Entries within a symptom class that share a superscript do not differ significantly from each other at (=0.05, | | | | | |
| # as determined by using the Tukey-Kramer honestly significant difference (HSD) analysis. | | | | | |

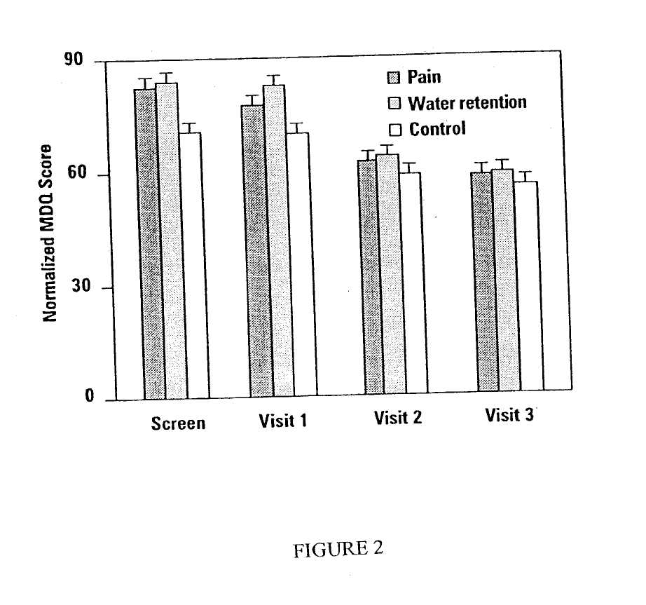

As assessed by the MDQ, subjects reported significant improvement (p<0.0002) in pain, water retention, negative affect, impaired concentration, and behavior change during the menstrual and premenstrual times after intervention with the medical food. Subjects reported significant improvement in negative affect and behavior change (p<0.005 and p<0.05, respectively) during the intermenstrual time as well. Improvement was also noted in autonomic reactions. The control symptoms showed some improvement, but not nearing the level of significance of those of the other categories (Table 10, FIG. 2), whereas little change was reported for the arousal symptoms category.

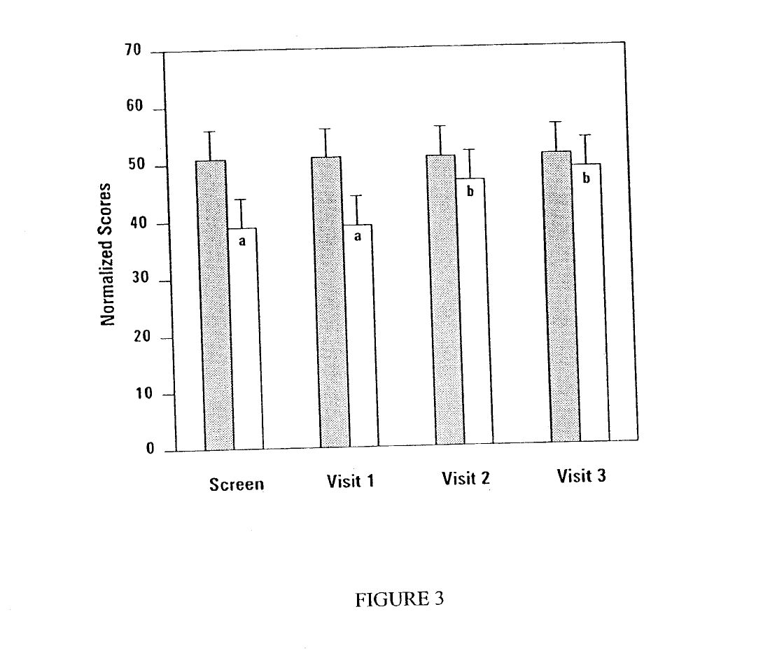

The SF-36 quality-of-life assessment reports general health and well-being as two scores: the Physical Component Score (PCS), an indication of physical pain and ability to function; and the mental Component Score (MCS), an indication of mood and affect. The PCS and MCS are normalized to 50, which is the average score observed in a healthy population. At initiation of the trial, the subjects rated 51.2 (±1.2) on the PCS, which remained constant throughout the trial (p=0.9773). The initial MCS scores were 38.8 (±1.6) and 38.9 (±1.6) for the Screening Visit and Visit 1, respectively, well below the mean, suggesting compromised mental well-being at initiation of the trial; the MCS scores were significantly increased by the end of the trial to 47.0 (±1.5) and 48.5 (±1.4; p<0.0001) for Visit 2 and Visit 3, respectively. These results are graphically depicted in FIG. 3.

The total excretion of estrogen metabolites, as assessed by a 24-hour urinary excretion of estrone, estradiol, and estriol was significantly increased after the intervention with the medical food (p<0.005) when data from all subjects were analyzed (Table 11). When total estrogen excretion was analyzed using the geometric mean (90% confidence), an increase was observed from 49.3 (43.1-56.5) μg/24 hours initially to 69.7 (59.4-81.7) μg/24 hours after the intervention with the medical food. Some beneficial changes were noted in serum steroid hormone metabolism markers as well, such as a decrease in HCys and testosterone and an increase in progesterone, but when data from all subjects were analyzed no significant changes were observed.

TABLE 11
Serum and urinary markers associated with hormone transport,
metabolism, and excretion for all subjects who completed the trial

| | Reference | Mean (± sem) | | |
|---|---|---|---|---|
| | Range | Baseline | Final | p |
| Homocysteine | <9.0 | 7.3 (0.3) | 6.6 (0.2) | 0.07 |
| (μmol/L)* | | | | |
| Total testosterone | 15-70 | 28.6 (2.1) | 28.5 (1.9) | ns |
| (ng/dL)* | | | | |
| Free testosterone | 1.0-8.5 | 4.2 (0.4) | 3.8 (0.3) | ns |
| (pg/mL)* | | | | |
| Progesterone | 0.2-28 | 8.8 (1.3) | 11.4 (1.6) | ns |
| (ng/mL)* | | | | |
| SHBG (nM)* | 17-120 | 82.2 (11.0) | 81.4 (10.2) | ns |
| Bound estradiol | 60-130 | 58.8 (8.7) | 65.3 (9.0) | ns |
| (pg/mL)* | | | | |
| Excreted estradiol | 18-162 | 53.5 (4.0) | 77.6 (6.6) | <0.005 |
| (μg)#,§ | | | | |

*N = 39;
#N = 35;
§Normative data are for estrogen excretion during the luteal phase.
Total estrogen excretion includes estrone, estradiol, and estriol excreted over 24 hours.

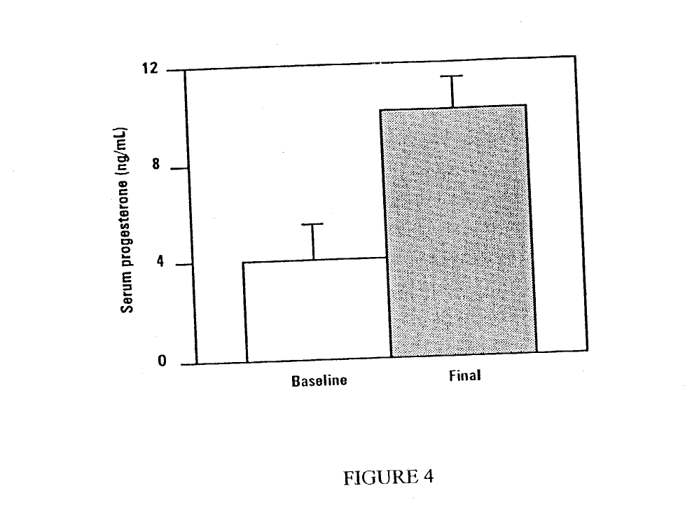

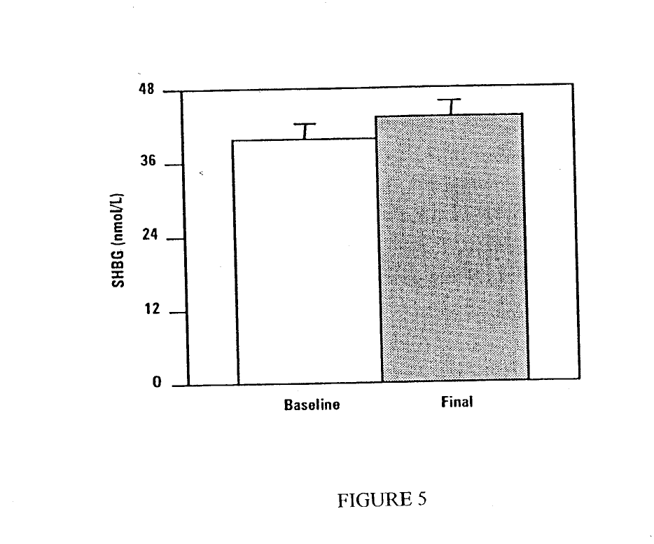

Although no significant changes in serum markers were noted when all data were analyzed, when the data were stratified based upon whether the subject showed initial values near the limit or outside of the normative range, significance was established, as shown in Table 12. Twenty-eight women presented with low bound estradiol, as compared to the reference range (<60 pg/mL); a significant increase in bound estradiol to 63.7 (±10.3) pg/mL was observed in these women after the intervention (p=0.002). The 16 women who presented with elevated unbound testosterone, defined as >1.5% free testosterone, showed a statistically significant decrease in serum testosterone (p<0.001). The 26 women with low initial serum progesterone, (<10 ng/mL), responded to the intervention with a statistically significant increase in serum progesterone to 10.2 (±2.01) ng/mL (p<0.005; FIG. 4). Likewise, the 12 women with elevated HCys (>8 mol/L; FIG. 5) at the start of the trial responded with a statistically significant decrease in serum HCys (p<0.001). SHBG also showed an increase from pre- to post-intervention in the 20 individuals who had initially low values (<5.5 nmol/L) from 39.9 (±2.0) to 43.3 (±2.7) nmol/L, respectively, but the increase was not statistically significant.

TABLE 12
Mean (± sem) serum hormone metabolites of subjects
for whom initial laboratory values were either at
the limits of, or not within reference range

| | Criterion | | N | Baseline | Final | p |
|---|---|---|---|---|---|---|
| High | >1.5% | | 16 | 1.90 (0.09) | 1.53 (0.04) | <0.001 |
| free | | | | | | |
| testos- | | | | | | |
| terone | | | | | | |
| Low | <10 | ng/mL | 26 | 4.1 (0.44) | 10.2 (2.01) | <0.005 |
| proges- | | | | | | |
| terone | | | | | | |
| Low | <55 | nmol/L | 20 | 39.9 (2.0) | 43.3 (2.7) | 0.07 |
| SHBG | | | | | | |
| Low | <60 | pg/mL | 28 | 31.3 (2.7) | 63.7 (10.3) | 0.002 |
| bound | | | | | | |
| estra- | | | | | | |
| diol | | | | | | |
| High | >8 | μmol/L | 12 | 9.4 (0.4) | 7.3 (0.3) | <0.001 |
| homo- | | | | | | |
| cys- | | | | | | |
| teine | | | | | | |

Data are provided for Baseline (prior to medical food intervention) and Final (after two months of medical food intervention) values, in addition to the criterion used to select data for each analysis.

Discussion

A preliminary study was conducted to assess the effects of a medical food of Example 1 for nutritional support for symptoms related to hormone cycles, with or without the phytonutrient 13C, over 2 complete menstrual cycles on PMS symptomatology. The primary endpoint for this study was subjective improvement of PMS as determined by 2 well-validated PMS symptoms-specific questionnaires; the SPAF and the MDQ. The results of the SPAF and MDQ suggest that consumption of the medical food of Example 1 nutritionally supported significant improvement in PMS-specific symptoms, such as pain, water retention, affect and mood. Furthermore, quality-of-life data and laboratory markers, such as total estrogen excretion, serum progesterone and testosterone, also showed significant improvement over the course of the intervention. These observations suggest that the medical food of Example 1 nutritionally supports metabolic changes in hormone metabolism that are associated with improvement in PMS symptomatology.

Data from subjects on and not on oral contraceptives were pooled due to failure to find distinction. Data between the 2 groups in the trial, the medical food/13C and medical food/placebo group, were also pooled since no differences in the primary end-points were noted between the 2 groups. The inability to distinguish between the 2 treatment groups argues only that 13C treatment had no additional effect on the resolution of PMS symptoms over that of the medical food alone. Data on estrogen metabolism suggests differences did occur in estrogen metabolites with the 13C and, consistent with published literature, that inclusion of 13C with the medical food can promote higher levels of the safer estrogenic metabolite, 2-hydroxyestrone (20H-E). The role of the estrogenic metabolites, such as 20H-E, in etiology or enhancement of symptoms remains unclear; however, 20H-E is considered a safer estrogen because higher levels of 20H-E are associated with a decrease in risk of hormone-dependent cancers, such as breast cancer.

One hypothesis for the biochemical imbalance underlying PMS symptomatology is an imbalance in the activity of estrogen to progesterone. This relative increase in estrogen activity has been termed estrogen dominance. High estrogen activity can be due to a low level of overall excretion of the estrogen metabolites, a decrease in SHBG with a high serum (free) levels of estrogen, and/or an increase in the more estrogenic metabolites over the less estrogenic metabolites. The medical food of the preferred embodiments was designed, in part, to nutritionally support an increase in estrogen excretion by providing fibers that preferentially bind sex hormones, including estrogen. Fiber can also facilitate excretion of estrogen by its effect on increasing transit through the colon. Data on estrogen excretion suggests that consumption of the medical food did result in a significant increase in excretion of estrone, estriol, and estradiol in the subjects on the trial (p<0.005).

The amount of estrogen and testosterone available to cells is influenced by the amount of SHBG present in circulation. SHBG can bind free estrogen or testosterone and, while bound, these hormones are not active. About half of the circulating testosterone and approximately 80% to 90% of circulating estrogen is bound to SHBG under optimal conditions. SHBG is produced in the liver, and its production is regulated by steroidal and peptidic hormones, and by dietary factors. In particular, dietary isoflavones and lignans have been shown to significantly increase the production of SHBG. In this study, consumption of the medical food resulted in an increase in SHBG levels in those individuals who initially presented with the lowest levels of SHBG (p=0.07). A moderate, but non-significant decrease in free testosterone was noted when data from all subjects were analyzed, whereas no change in serum testosterone was observed; however, a significant decrease in free testosterone was observed when the data from subjects who presented with the highest levels of free testosterone were reviewed (p<0.001). A significant increase in bound estradiol was also observed in the 28 women who presented with low bound estradiol (p=0.002). Taken together, these observations suggest that SHBG levels were increased as a result of the medical food intervention.

One pathway for metabolism of the estrogen metabolites involves methylation by the catechol-O-methyltransferase enzyme, which uses the methyl-donor SAM. The methylated estrogens show low estrogenic activity, are considered anti-estrogenic, and are rapidly excreted. The methylated estrogen derivative of 20H-E has been shown to inhibit the growth of breast cancer cells, have antiangiogenic activity, and inhibit adipocyte proliferation, suggesting it may be a protective estrogen. Thus, nutritional support for production of SAM, and therefore for methylation itself, may positively influence estrogen metabolism. Nutrients that support SAM production included in the medical food of the preferred embodiments are vitamins B6, B12, and folate, as well as choline and trimethylglycine. It is unknown whether these nutrients resulted in an increase in methylation of estrogen in this trial; however, a quarter of the subjects presented with high circulating HCys levels, which is an indication of compromised methylation. The level of HCys was significantly decreased over the course of the intervention in these subjects (p<0.001), suggesting that methylation was improved.

Estrogen dominance can occur when estrogen metabolism is normal and progesterone production is low. Over about half of the subjects in the trial presented with low or low-normal initial serum progesterone levels, and the serum progesterone was significantly increased over the course of the intervention in these subjects (p<0.005). Few data have been reported on the role of nutritional support for progesterone production, and its role in PMS symptomatology is controversial. For example, although the most popular theory of hormone involvement in PMS symptoms implicates low progesterone during some phase of the cycle, placebo-controlled trials with progesterone supplementation have not unequivocally ameliorated symptoms and, therefore, have not supported this hypothesis. Thus, it would appear that estrogen makes PMS symptoms worse.

In contrast to the observations that high levels of estrogen are associated with more intense PMS symptoms, estrogen supplementation has been shown to attenuate PMS symptoms. Therefore, the role of estrogen and progesterone in PMS symptomatology is unclear. A factor is not just the absolute levels themselves, but the ratio of estrogen to progesterone, and possibly the nature of the estrogen metabolites within this ratio. The observed increase of progesterone in individuals who initially displayed the lowest serum progesterone levels could have resulted in reestablishment of a more balanced, beneficial estrogen-to-progesterone ratio. Alternatively, increases in serum progesterone may have occurred from an increase in ovulatory cycles, which can also affect the ratio of estrogen to progesterone in the luteal phase of the menstrual cycle.

PMS symptoms show a strong placebo effect. The preliminary clinical trial reported in this Example did not contain a control group, and therefore, placebo effect should be considered in evaluating these data. The MDQ contains a control category that allows an estimation of placebo effect, since it reflects symptoms not generally associated with PMS that should be equally responsive to placebo as PMS-specific symptoms. There was some change in symptoms in the control category of the MDQ. The MDQ control category includes the symptoms of chest pains, feelings of suffocation, ringing in the ears, heart pounding, numbness and tingling, and effects on vision. Although these symptoms are not generally associated with PMS, some of them are associated with early perimenopause, which has similar hormonal fluctuations as PMS. The overlap of symptoms can explain why a significant change was observed in this category for menstrual and premenstrual symptoms (p<0.03). However, this change was not as highly significant as the changes in pain, water retention, affect, concentration, and behavior for menstrual and premenstrual symptoms (p<0.0001). Moreover, laboratory markers show significant changes, which would be unlikely to result from a placebo effect alone. Therefore, taken together, these data are fully concordant and suggest that the medical food, via nutritional modulation of hormone metabolism, significantly reduces PMS symptoms.

### EXAMPLE 4

Clinical Study of the Effects of Medical Composition in the Form of Tablet

The study was performed at the Functional Medicine Research Center, Gig Harbor, Wash. from January to May 2002.

Potential subjects were recruited through newspaper and radio advertisements. Women aged 40 to 65 years with either 6 months of amenorrhea and a biochemical criterion for menopause (i.e., FSH greater than 50 mIU/mL, estradiol less than 20 pg/mL), or 12 months of amenorrhea with or without biochemical criterion for menopause were accepted for the trial. Subjects younger than 40 were eligible to participate only if they had had a complete bilateral ovariectomy more than 6 weeks prior to the start of the trial. Subjects had to be experiencing greater than, or equal to, 40 hot flushes and/or night sweats per week (6/day).

Individuals were excluded from participating in the trial if they had evidence of: untreated endocrine, neurological, or infectious disorder; pregnancy or lactation; history of diabetes; mental illness, or attempted suicide; liver, kidney, or heart disease; use of oral corticosteroids within 4 weeks prior to screening; use of oral birth control medication, oral estrogens, or estrogen-, progestin-, or progesterone-containing creams or patches; active cancer or a personal history of cancer (excluding skin cancer), use of a supplement containing isoflavones in proceeding 4 weeks; or evidence of HIV. The initial screening visit included a laboratory assessment for abnormal CBC, glucose, kidney or liver function.

Clinical Study Ethics

The study protocol for this clinical trial was approved by the Washington Institutional Review Board (Olympia, Wash.). Candidates who agreed to participate signed Informed Consents.

Study Design

The clinical trial was a single-arm, open-label, observational study. All subjects completed a 2-week run-in period, in which they kept daily records of the number and intensity of hot flushes and night sweats. At 2 weeks they returned to start the active phase of the trial. Subjects who showed an average of less than 40 hot flushes and night sweats per week during the run-in period were disqualified from participation.

Each subject was dispensed a 90-count bottle of the nutritional supplement and were instructed to take 3 tablets once a day with food. The ingredients in the supplement are shown in Table 13. Subjects were also counselled to maintain their customary dietary and lifestyle patterns. Diet and lifestyle habits were monitored on questionnaires initially, and at weeks 6, 10, and 14, to identify any changes. Blood pressure, pulse and weights were collected at screening, 2, 6, 10 and 14 weeks. Compliance was calculated by tablet count of the returned containers.

TABLE 13
List of Ingredients in Nutritional Supplement of Clinical Study

| Micronutrients | Amount per day | |
|---|---|---|
| Vitamin A (mixed carotenoids and palmitate) | 2500 | IU |
| Vitamin D | 200 | IU |
| Vitamin E | 200 | IU |
| Vitamin K | 40 | mcg |
| Vitamin B6 | 50 | mg |
| Vitamin B12 | 30 | mcg |
| Folic Acid | 400 | mcg |
| Isoflavones from red clover (Trifolium pratense) | 50 | mg |
| Isoflavones from kudzu (Pueraria lobata) | 50 | mg |
| Curcumin | 200 | mg |
| Trimethylglycine | 200 | mg |
| Resveretrol | 2 | mg |
| Rosemary extract | 200 | mg |
| Chrysin | 100 | mg |
| 5-methyltetrahydrofolate | 400 | mcg |

Other ingredients:
Microcrystalline cellulose, croscarmellose sodium, stearic acid, calcium silicate, silica, and magnesium stearate.

The primary endpoint assessed was change in frequency and intensity of hot flushes and night sweats as self-reported on daily symptom records. To determine effect on hot flush and night sweats, the average of the two-week control period was taken as baseline and compared to the average of the last two weeks of the treatment period.

Secondary clinical endpoints included assessment of subjective improvement of menopausal symptoms as measured by the Greene Climacteric Questionnaire. The Greene Questionnaire is a standardized menopause-specific instrument, which measures symptoms of the climacteric including hot flushes and night sweats. Questionnaire data were collected at screening, 2, 6, 10, and 14 weeks.

Fasting blood samples were taken at the beginning and end of the study to assess for changes in liver and kidney function, glucose, and complete blood count (CBC). Additional laboratory tests included follicle stimulating hormone (FSH), (taken at the beginning of the trial only), estrogen metabolites (estrone [E1], estradiol [E2], estriol [E3], 2-hydroxyestrone [2-OHE1], 16alpha-hydroxyestrone [16alpha-OHE1]), progesterone, testosterone, sex hormone binding globulin (SHBG), dehydroepiandrosterone-sulfate (DHEA-S), homocysteine, blood lipids, and isoflavones.

Analytical Methods

Aspartate aminotransferase, alanine aminotransferase, bilirubin, urea nitrogen, creatinine, albumin, and glucose were assessed by standard photometric methods; CBC was assessed by Coulter GenS; FSH and E2 were assessed by chemiluminescence; triglycerides, total-, HDL-, and LDL-cholesterol were determined by photometric analysis, and homocysteine was assessed by high performance liquid chromatography at Laboratories Northwest (Tacoma, Wash.). Radioimmunoassay measurements of SHBG, progesterone, testosterone, DHEA-S, E1, E2 and E3, and ELISA-colorimetric analysis of 2-OHE1 (2-OHE) and 16alpha-OHE1 (16-OHE) were performed by Great Smokies Diagnostic Laboratories (Asheville, N.C.). GC/Mass Spectrometry measurements of the isoflavones (daidzein, genistein, equol, gycetitin, O-MDA, formononetin, and biochanin A) were performed in the laboratory of Kenneth D. R. Setchell PhD, Children's Hospital Medical Center (Cincinnati, Ohio).

Statistical Analysis

Data were analyzed by a one-way analysis of variance (ANOVA) using JMP Statistical Package (SAS Institute, Cary, N.C.). Variances in laboratory analyses were determined using multiple split samples. Average values are presented as mean±sem.

Results

One hundred and eighty women were screened initially; of these, 31 women were accepted for the study. Twenty-five of the 31 subjects (average age 53 years) completed the trial. Six subjects dropped out of the trial before completion: five because of an inability to comply with the study protocol and one because the subject did not fit hot flush criteria after the initial 2-week run-in period. All subjects on screening had normal CBC, serum glucose and liver/kidney function. No statistically significant changes in the screening laboratory tests were noted at the conclusion of the trial. No significant changes in weight and blood pressure were observed throughout the trial.

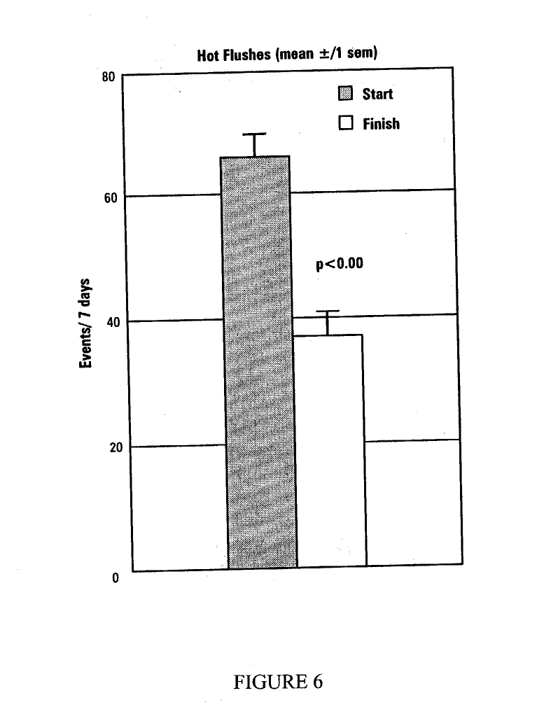

Both the frequency and intensity of hot flushes and night sweats decreased significantly when the initial run-in values were compared to the occurrence during the last two weeks of the 12-week intervention. Frequency decreased from an average of 68±5 flushes per 7 days initially to 37±6 flushes per 7 days at the end of the trial, for an average decrease of 46% (p<0.001; FIG. 6).

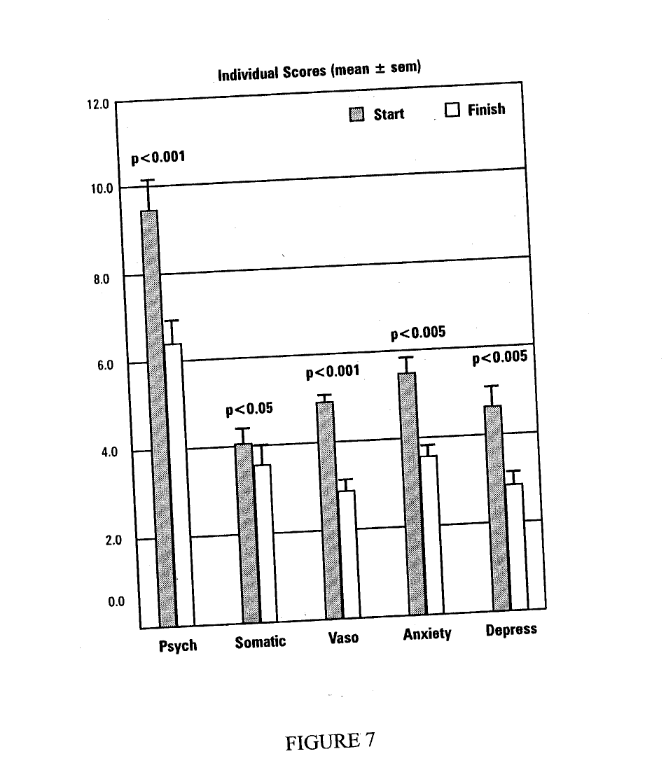

The data obtained from the Greene Questionnaire also supported this observation. The category of vasomotor symptoms on the Greene Questionnaire significantly decreased from a score of 4.8±0.2 to 3.1±0.3 (p<0.001). As can be seen in FIG. 7, all categories of the Greene Questionnaire, including psychological, somatic, anxiety, and depression, significantly decreased, and the overall score was significantly reduced from 20±1.4 to 14±1.4 (p<0.001).

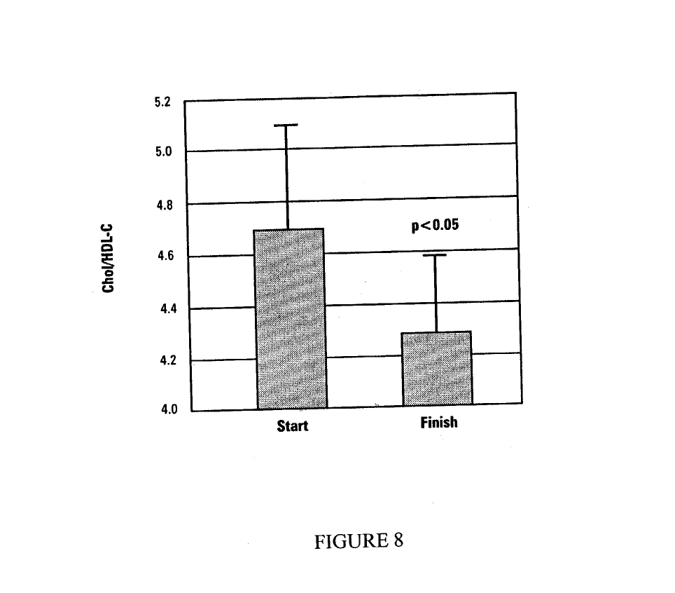

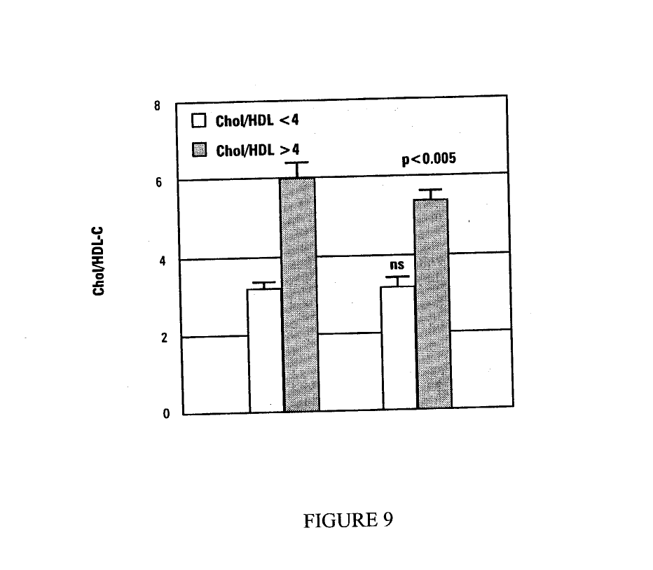

The cardiovascular risk markers homocysteine and total cholesterol-to-HDL-chol ratio also showed significant decrease. While total cholesterol did not significantly decrease over the course of the intervention, the Chol/HDL-C ratio decreased from 4.71±0.35 to 4.32±0.29 (p<0.03), for an overall decrease of 8% among all participants. The decrease in the ratio was even greater when the participants were stratified between those subjects who initially started with a ratio >4 (13% decrease) (FIGS. 8 and 9).

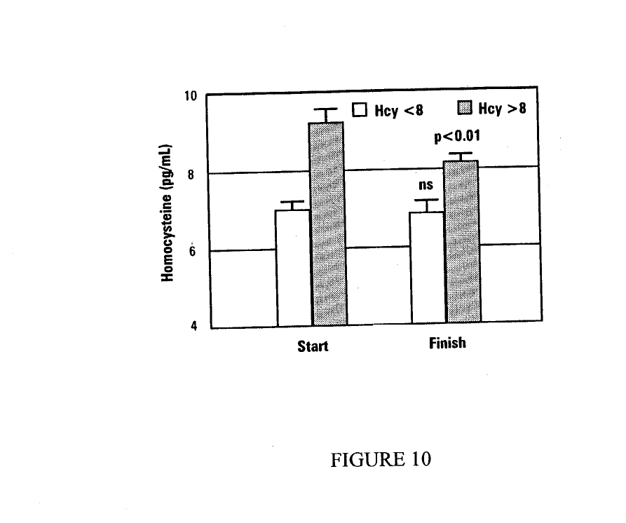

Homocysteine significantly decreased from an initial average of 8.29±0.32 pg/mL to 7.51±0.25 pg/mL (p<0.005). However, when the homocysteine data are analyzed only for those subjects who initially presented with elevated homocysteine, the resulting data are more dramatic. Initially, 14 of the subjects had homocysteine values above 8.0 pg/mL, and 8 of these subjects had values above 9.0 pg/mL. After the intervention, homocysteine values had reduced to below 9.0 pg/ml in all but one subject, and 7 of these had reductions to 8.0 pg/mL or lower (FIG. 10).

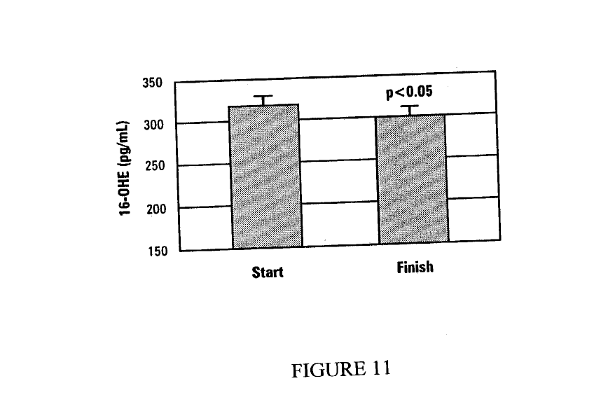

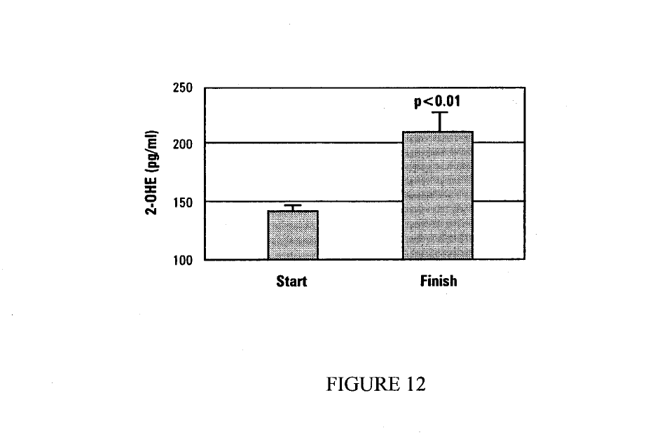

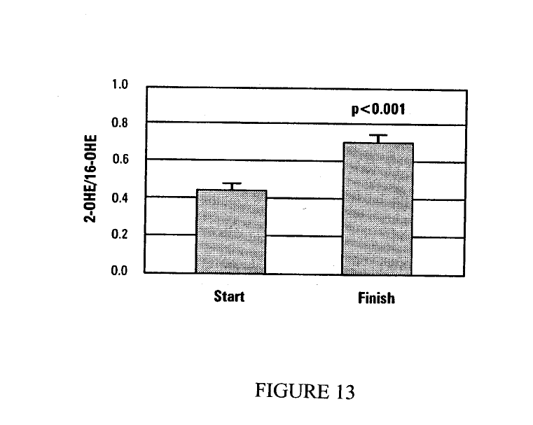

The serum for 2-OHE and 16-OHE was analyzed. As shown in FIGS. 11 and 12, initial 2-OHE and 16-OHE were 140±6.20 (pg/ml) and 315±11.0 (pg/ml), respectively. After the intervention with the nutritional supplement, 2-OHE was significantly increased to 209±13.7 (pg/ml) (p<0.01), whereas 16-OHE was significantly decreased to 296±13.7 (pg/ml) (p<0.05). The change in these values resulted in a significant increase in the ratio of 2:16 from 0.46±0.024 initially to 0.71±0.063 (p<0.001); a 35% increase (FIG. 13).

Discussion

Women who reach menopause face a number of issues. Although many come to their doctor seeking treatment for climacteric symptoms, longer-term issues of heart disease and breast cancer are often part of the anxiety. The negative results from the most recent prospective trials using HRT in postmenopausal women have put many of these women in a quandary. As it appears that HRT has failed to achieve the early promise of success that they were once thought to provide, women are searching for other avenues that address these concerns.

This is the first trial to look at the effects of a combination of isoflavone product made with kudzu and red clover for the remediation of hot flushes.

During the 12-week intervention with the nutritional supplement, we observed a significant decrease in reported hot flushes from an average of 9.7 per day to 5.2 per day. Quality of life, as assessed by the Greene Questionnaire also improved. All categories on the Greene Questionnaire individual subscales (psychological, somatic, vasomotor, anxiety and depression) showed statistically significant improvement.

Several markers of cardiovascular disease risk also showed improvement over the 12-week intervention. The Chol/HDL-chol ratio improvement may also be attributed to the isoflavones in the product. Isoflavones have been shown in some, but not all studies, to exert a mild improvement in the Chol/HDL-chol ratio.

Serum levels of homocysteine also improved. Homocysteine was decreased on average 9% in the whole group. In those women who started with elevated levels (defined as greater than 8 pg/mL) the decrease was even more significant at 13%. Epidemiological studies have shown that higher blood homocysteine levels appear to be associated with higher risks of coronary, cerebral, and peripheral vascular disease and are inversely related to blood levels of folate, vitamin B₁₂ and B₆ Additionally, there is some research to suggest these vitamins may be important in breast cancer risk as well. Catechol-O-methyltransferase (COMT) catalyzes the O-methylation of catechol estrogens. Several studies have indicated that COMT polymorphisms, which results in a three- to four-fold decrease in activity, is associated with increased breast cancer risk. These findings indicate a role for certain folate pathway micronutrients in mediating the association between COMT genotype and breast cancer risk.

We observed a significant increase in the ratio of 2-OHE1 to 16alpha-OHE1. Research suggests that women who metabolize a larger proportion of their estrogens through the C-16 pathway, as opposed to the C-2 pathway, have an elevated breast cancer risk. In one recent large trial of 10,786 premenopausal women followed for 5.5 years, it was found that participants with increased levels of 2-OHE had a 40 percent decrease in the occurrence of breast cancer. In a longer-term study on postmenopausal women, those with the highest 2-OHE:16-OHE ratio had 30 percent less risk of developing breast cancer than women with lower ratios. While not all studies have been positive, the data overwhelmingly favors that a higher 2-OHE level is beneficial, especially in postmenopausal women at risk for hormone-dependent cancer.

While much of the research on 2:16 ratio has focused on the phytonutrient indole-3-carbinol—which is found in cruciferous vegetables—the increase we saw in our trial may be due to the isoflavones. In studies on both pre- and postmenopausal women, it has been shown that isoflavones increase the beneficial 2-OHE at the expense of the 16-OHE, resulting in an increased 2:16 ratio. Moreover, it may be that the specific isoflavones found in kudzu have the most pronounced effect. One of kudzu's isoflavones, puerarin, induces the cytochrome P450 enzymes 1A1 and 1A2; these enzymes are instrumental in increasing 2-hydroxylation of estrogens. Additionally, preliminary research suggests the herb rosemary (Rosmarinus officinalis), also an ingredient of the supplement, may also promote 2-hydroxylation of estrogen, and may support an increased 2:16 ratio.

Although our trial suffered from lack of a control group, the approximately 50% improvement in symptoms agrees with most of the published studies using soy and red clover isoflavones. Therefore, this observational study suggests that this nutritional supplement may have a salutatory effect on hot flushes and night sweats. In addition, a modest but statistically significant improvement in 2-OHE:16-OHE ratio, total Chol/HDL-chol ratio, and homocysteine, suggests that this combination nutritional formula may potentially confer not only symptomatic but some chemopreventive and cardioprotective effects for women beginning menopause. A rigorous, placebo controlled trial to follow up on these observations is in order.

Many modifications and variations of the embodiments described herein may be made without departing from the scope, as is apparent to those skilled in the art. The specific embodiments described herein are offered by way of example only. Further information which those skilled in the art will find useful when implementing embodiments of the present invention can be found in the materials attached hereto as an Appendix and which are now herein incorporated by reference in their entireties as well as all of the publications cited therein.

What is claimed is:

1. A method of managing a bodily process that utilizes S-adenosylmethionine (SAM) in a pathway of the bodily process comprising administering a composition comprising a mixture of an isoflavone, an isoflavone synergist, and a methylation support compound.

2. The method of claim 1, wherein the bodily process is hormone imbalance.

3. The method of claim 2, wherein the pathway is methylation of estrogenic metabolites.

4. The method of claim 3, wherein the pathway is catalyzed by catechol O-methyltransferase (COMT).

5. The method of claim 3 wherein the pathway is catalyzed by S-adenosyl-L-methionine:delta-24[25]sterol methyltransferase.

6. The method of claim 1, wherein the pathway is methylation of a compound selected from the group consisting of catecholamines, neurotransmitters, proteins, membrane phospholipids, fatty acids, nucleic acids, porphyrins, choline, carnitine, creatine, and hormones.

7. The method of claim 6, wherein the hormone is selected from the group consisting of peptide hormone, amine hormone, steroid hormone, and eicosanoid.

8. The method of claim 1, wherein the pathway is DNA methylation.

9. A method of treating or preventing a condition or disease involving a bodily process that utilizes S-adenosylmethionine (SAM) in a pathway of the bodily process comprising administering a composition comprising a mixture of an isoflavone, an isoflavone synergist, and a methylation support compound.

10. The method of claim 9, wherein the bodily process is hormone imbalance.

11. The method of claim 10, wherein the condition or disease is selected from the group consisting of cardiovascular disease, hot flushes, cancer, premenstrual syndrome, endometriosis, uterine fibroid tumors, fibrocystic or painful breasts, cervical dysplasia, systemic lupus erythematosis, vaginitis, fatigue, cognitive dysfunction, depression, and irritability.

12. The method of claim 10, wherein the condition or disease is treated or prevented through a mechanism selected from the group consisting of promoting C-2 hydroxylation over C-4 and/or C-16 hydroxylation of estrogen and estrogenic metabolites, reducing oxidation of catechol estrogens (2-OH and 4-OH), increasing circulating concentrations of sex hormone binding globulin (SHBG), inhibiting activity of aromatase, and upregulating Phase I and Phase II liver enzymes.

13. The method of claim 12, wherein the composition that is administered further comprises a component selected from the group consisting of cruciferous vegetables, indole-3-carbinol, vitamin A, vitamin E, vitamin C, N-acetylcysteine, turmeric, green tea, lycopene, Ε-lipoic acid, flavonoids, folate, vitamin B2, vitamin B6, vitamin B12, trimethylglycine, magnesium, fiber, lignans, d-limonene, probiotics, and calcium D-glucarate.

14. The method of claim 9, wherein the condition or disease is selected from the group consisting of cancer, liver damage, brain cell degeneration, depression, osteoporosis, fibromyalgia, gastrointestinal injury, liver dysfunction, migraine, Parkinson's disease, Alzheimer's disease, organic brain syndrome, epilepsy, HIV-related neurologic complications, multiple sclerosis, metabolic defects, and spinal cord disease.

15. The method of claim 1, wherein the isoflavone is derived from a food source selected from the group consisting of kudzu root, soy, legumes, alfalfa, clover, and licorice root.

16. The method of claim 1, wherein the isoflavone is derived from kudzu.

17. The method of claim 1, wherein the isoflavone synergist is a ingredient selected from the group consisting of curcumin, rosemary extract, and resveratrol.

18. The method of claim 1, wherein the methylation support compound is an ingredient selected from the group consisting of choline, trimethylglycine, cobalamin and derivatives thereof, and folic acid and derivatives thereof, riboflavin, pyridoxine, and magnesium.

19. The method of claim 1, further comprising at least one ingredient selected from the group consisting of vitamin, mineral, fortifying amino acid, carotenoid, and flavonoid.

20. The method of claim 19, wherein the vitamin is at least one vitamin selected from the group consisting of vitamin A, vitamin D, vitamin E, vitamin K, thiamin, riboflavin, niacin, pyridoxine, pantothenic acid, biotin, vitamin C, and derivatives thereof.

21. The method of claim 19, wherein the mineral is at least one mineral selected from the group consisting of calcium, magnesium, chromium, copper, iodine, iron, phosphorus, molybdenum, selenium, zinc, manganese, sodium, and potassium.

22. The method of claim 19, wherein the fortifying amino acid is at least one amino acid selected from the group consisting of L-lysine, L-threonine, and N-acetylcysteine.

23. The method of claim 19, wherein the carotenoid is at least compound selected from the group consisting of lutein, zeaxanthin, β-carotene, and lycopene.

24. The method of claim 19, wherein the flavonoid is at least compound selected from the group consisting of quercetin, chrysin, and hesperidin complex.

25. A method of treating a female mammal experiencing hot flushes comprising administering to said mammal a composition comprising a mixture of an isoflavone, an isoflavone synergist, and a methylation support compound.

26. The method of claim 25, wherein the isoflavone is derived from a food source selected from the group consisting of kudzu root, soy, legumes, alfalfa, clover, and licorice root.

27. The method of claim 25, wherein the isoflavone is derived from kudzu.

28. The method of claim 25, wherein the isoflavone synergist is a ingredient selected from the group consisting of curcumin, rosemary extract, and resveratrol.

29. The method of claim 25, wherein the methylation support compound is an ingredient selected from the group consisting of choline, trimethylglycine, cobalamin and derivatives thereof, and folic acid and derivatives thereof, riboflavin, pyridoxine, and magnesium.

30. The method of claim 25, further comprising at least one ingredient selected from the group consisting of vitamin, mineral, fortifying amino acid, carotenoid, and flavonoid.

31. The method of claim 30, wherein the vitamin is at least one vitamin selected from the group consisting of vitamin A, vitamin D, vitamin E, vitamin K, thiamin, riboflavin, niacin, pyridoxine, pantothenic acid, biotin, vitamin C, and derivatives thereof.

32. The method of claim 30, wherein the mineral is at least one mineral selected from the group consisting of calcium, magnesium, chromium, copper, iodine, iron, phosphorus, molybdenum, selenium, zinc, manganese, sodium, and potassium.

33. The method of claim 30, wherein the fortifying amino acid is at least one amino acid selected from the group consisting of L-lysine, L-threonine, and N-acetylcysteine.

34. The method of claim 30, wherein the carotenoid is at least compound selected from the group consisting of lutein, zeaxanthin, β-carotene, and lycopene.

35. The method of claim 30, wherein the flavonoid is at least compound selected from the group consisting of quercetin, chrysin, and hesperidin complex.
